(19) United States
(12) Patent Application Publication
(10) Pub. No.: US 2023/0265493 A1
(43) Pub. Date: Aug. 24, 2023

(54) AORTA-SPECIFIC DNA METHYLATION PATTERNS IN CELL-FREE DNA FROM PATIENTS WITH BICUSPID AORTIC VALVE-ASSOCIATED AORTOPATHY

(71) Applicant: UTI Limited Partnership, Calgary (CA)

(72) Inventors: Steven GREENWAY, Calgary (CA); Paul W.M. FEDAK, Calgary (CA); Ashna Karimbhai MAREDIA, Calgary (CA); Mohammad ALEINATI, Calgary (CA)

(21) Appl. No.: 18/067,247

(22) Filed: Dec. 16, 2022

Related U.S. Application Data
(60) Provisional application No. 63/290,949, filed on Dec. 17, 2021.

(30) Foreign Application Priority Data
Dec. 17, 2021 (CA) 3,142,851

(51) Int. Cl. C12Q 1/686

(52) U.S. Cl. CPC C12Q 1/686; C12Q 2600/154; C12Q 2600/118; G01N 2800/32

(57) ABSTRACT

Described herein is a method of determining the risk of a human subject with congenital BAV having BAV-associated aortopathy or developing BAV-associated aortopathy, comprising: obtaining a cell free DNA (cfDNA) sample from the subject, measuring the level of methylation of a methylation marker that is associated with BAV-associated aortopathy in the cfDNA sample, optionally comparing the level of methylation of the methylation maker in the sample of the subject with a comparator control, wherein when the methylation marker is hypo- or hypermethylated the subject as being at risk of developing BAV-associated aortopathy.

### SEQUENCE LISTING

This application contains a Sequence Listing which has been filed electronically in Extensible Markup Language (XML) format and is hereby incorporated by reference in its entirety. Said XML, created on Dec. 14, 2022, is named 51012-053001_Sequence Listing 12_14_22.XML and is 8,049 bytes in size.

### FIELD

The present disclosure relates generally to a method of determining the risk of a human subject with congenital BAV having BAV-associated aortopathy or developing BAV-associated aortopathy.

### BACKGROUND

The aortopathy that occurs as a consequence of a congenitally bicuspid aortic valve (BAV) is associated with a risk of dissection, aneurysm or rupture (1). The etiology of BAV-associated aortopathy appears to be multifactorial and related to inherent genetic defects (2) combined with hemodynamic wall shear stress (WSS) created by turbulence across the abnormal valve (3-6). Increased aortic WSS leads to the degradation of elastin and the dysregulation of extracellular matrix proteins which are linked to smooth muscle cell death (7-9). With progressive aortopathy, aortic valve replacement surgery is often recommended but patient selection is controversial and often dependent upon individual and institutional practice (10-14). A blood-based assay, potentially in combination with advanced imaging techniques, to identify those who would most benefit from prophylactic surgery would be a significant advance (15).

### SUMMARY

In one aspect there is provided a method of determining the risk of a human subject with congenital BAV having BAV-associated aortopathy or developing BAV-associated aortopathy, comprising:

obtaining a cell free DNA (cfDNA) sample from the subject,

measuring the level of methylation of a methylation marker that is associated with BAV-associated aortopathy in the cfDNA sample,

optionally comparing the level of methylation of the methylation maker in the sample of the subject with a comparator control,

wherein disproportionate hypomethylation of the methylation marker in the cfDNA sample would suggest that the subject is at risk of developing BAV-associated aortopathy.

In one example, the methylation marker comprises a differentially methylated region on Chr 11.

In one example, the DMR on Chr 11 comprises position Chr 11:3,168,734-3,168,832.

In one example, the step of measuring the cfDNA for the presence of one or more methylation markers comprising sequencing the cfDNA.

In one example, sequencing comprises bisulfite sequencing.

In one example, bisulfite sequencing is carried out using a forward primer (GGGTATTTAGTTATGAGGGAATAATG; SEQ ID NO:1) and a reverse primer (CAAACCTATCTTTAATTTCCACCC; SEQ ID NO:2).

In one example, the sequencing comprises droplet digital PCR assay.

In one aspect there is provided a kit for determining the risk of a human subject with congenital BAV having BAV-associated aortopathy or developing BAV-associated aortopathy, comprising:

a forward primer (GGGTATTTAGTTATGAG-GGAATAATG; SEQ ID NO: 1) and a reverse primer (CAAACCTATCTTTAATTTCCACCC; SEQ ID NO:2), optionally a container, and optionally instructions for the use there of.

### BRIEF DESCRIPTION OF THE DRAWINGS

Embodiments of the present disclosure will now be described, by way of example only, with reference to the attached Figures.

FIG. 1. Bioinformatics pipeline used to identify DMRs. Two separate comparisons were conducted in parallel, comparing a human aorta methylome to methylomes from 21 tissues and 14 types of hematopoietic cells to identify 446 and 181 DMRs, respectively. A total of 24 candidate aorta-specific DMRs were shared by both datasets.

FIGS. 2A-2C. FIG. 2A, Aortic wall cell death (indicated by percentage of TUNEL-positive cells) for 15 patients with varying maximal aortic diameters comparing regions of normal and elevated aortic WSS. Data presented are mean±SD with * indicating p<0.05 as determined by a Mann-Whitney U test. FIG. 2B, Paired patient data was found to be significantly different (p=0.00006) using the Wilcoxon signed-rank test. FIG. 2C, Summarized data. Data are mean±SD with significantly greater cell death in regions of elevated WSS as determined by a Mann-Whitney U test (p=0.0027).

FIGS. 3A-3D. FIG. 3A, Percentage of unmethylated CpG sites for the Chr 11 DMR in aorta and other human tissues and organs. FIG. 3B, Percentage of unmethylated CpG sites for the Chr 18 DMR in aorta and other human tissues and organs. FIG. 3C, Percentage of unmethylated CpG sites for the Chr 20 DMR in aorta and other human tissues and organs. FIG. 3D, Percentage of unmethylated CpG sites for the Chr 22 DMR in aorta and other human tissues and organs.

FIGS. 4A-4D. FIG. 4A, Aorta-specific cfDNA concentration as determined by the Chr 11 DMR for 23 patients sorted by maximal aortic diameter. FIG. 4B, Aorta-specific cfDNA concentration as determined by the Chr 18 DMR for 23 patients sorted by maximal aortic diameter. FIG. 4C, Aorta-specific cfDNA concentration as determined by the Chr 20 DMR for 23 patients sorted by maximal aortic diameter. FIG. 4D, Aorta-specific cfDNA concentration as determined by the Chr 22 DMR for 23 patients sorted by maximal aortic diameter.

FIGS. 5A-5D. FIG. 5A, Significant correlation (R² 0.59, p=0.0035) between levels of aorta-specific cfDNA as measured using the Chr 11 DMR and TUNEL staining in regions of elevated WSS. FIG. 5B, Significant correlation (R² 0.62, p=0.012) between levels of aorta-specific cfDNA as measured using the Chr 18 DMR and TUNEL staining in regions of elevated WSS. FIG. 5C, Non-significant correlation (R² 0.55, p=0.06) between levels of aorta-specific cfDNA as measured using the Chr 20 DMR and TUNEL staining in regions of elevated WSS. FIG. 5D, Significant correlation (R² 0.52, p=0.0078) between levels of aorta-specific cfDNA as measured using the Chr 22 DMR and TUNEL staining in regions of elevated WSS.

FIGS. 6A-6C. FIG. 6A, Aorta tissue and TUNEL staining. FIG. 6B, Aorta tissue and DAPI staining. FIG. 6C, Colocalization indicating the percentage of dying cells.

FIGS. 7A-7D. FIG. 7A, Total and aorta-specific cfDNA (as measured by the Chr 11 DMR) for patients with varying maximal aortic diameters. FIG. 7B, Total and aorta-specific cfDNA (as measured by the Chr 18 DMR) for patients with varying maximal aortic diameters. FIG. 7C, Total and aorta-specific cfDNA (as measured by the Chr 20 DMR) for patients with varying maximal aortic diameters. FIG. 7D, Total and aorta-specific cfDNA (as measured by the Chr 22 DMR) for patients with varying maximal aortic diameters.

FIGS. 8A-8D are graphs showing no significant correlation between levels of aorta-specific cfDNA as measured using the DMRs and TUNEL staining in regions of normal WSS. FIG. 8A) Chr 11 DMR cfDNA levels; FIG. 8B) Chr 18 DMR cfDNA levels; FIG. 8C) Chr 20 DMR cfDNA levels; and FIG. 8D) Chr 22 DMR cfDNA levels.

FIG. 9. No significant correlation between the total levels of cfDNA and TUNEL staining in regions of elevated WSS.

### DETAILED DESCRIPTION

Generally, the present disclosure provides a method of determining the risk of a human subject with congenital BAV having BAV-associated aortopathy or developing BAV-associated aortopathy, comprising: obtaining a cell free DNA (cfDNA) sample from the subject, measuring the level of methylation of a methylation marker that is associated with BAV-associated aortopathy in the cfDNA sample, optionally comparing the level of methylation of the methylation maker in the sample of the subject with a comparator control, wherein when the methylation marker is hypomethylated the subject as being at risk of developing BAV-associated aortopathy.

As used herein, the term “BAV” refers to Bicuspid Aortic Valve.

Generally, BAV refers to a cardiac congenital anomaly, in which two of the aortic valvular leaflets fuse, resulting in a valve that is “bicuspid” as opposed to the normal “tricuspid.”

As used herein the term BAV refers to any anatomical configuration in which two cusps are fused, irrespectively of the type of fusion. BAV can exist in isolation, but is often associated with other congenital cardiac lesions.

In some examples, an associated finding is dilation of the proximal ascending aorta secondary to abnormalities of the aortic media. Changes in the aortic media are present independent of whether the valve is functionally normal, stenotic, or incompetent. For this reason, BAV disease is considered a disease of both the valve and the aorta. Thus, BAV disease includes dysfunction of the tract of the aorta, including the ascending aorta, aortic arch, descending aorta, and abdominal aorta. BAV is associated with frequent and premature occurrence of life-threatening cardiac events, such as ascending aortic aneurysm and dissection, and significant valvular dysfunction, such as aortic stenosis (AS) and aortic insufficiency (AI).

As used herein, the term “BAV disease” or “BAV associated disease” includes both valvular and vascular pathologies associated with the detection of BAV.

“BAV disease” and “BAV syndrome” encompass both of these pathologies and may be used interchangeably.

The term “aortopathies” and “aortopathy”, as used herein, refer to any pathological condition of the aorta (for example dilatation, aneurysm, dissection, coarctation or simply dysfunction) whether determined by genetic diseases or having a sporadic onset. BAV disease can be a type of aortopathy.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

As used herein, the term“cell-free DNA” or “cfDNA” refers to non-encapsulated DNA in the blood and/or other bodily fluids. cfDNA generally are thought to enter the blood during apoptosis or necrosis. In a specific example, the cfDNA is from a blood sample from a subject.

The term “methylation marker” as used herein refers to any suitable biomarker based on one or more methylated bases in a nucleotide sequence which functions as an objective indication of a medical state, e.g., BAV-associated aortopathy.

In one example, one or more methylation markers may comprise differentially methylated regions (DMRs).

The term “differentially methylated region” or“DMR”, as used herein, refers to a category of methylation marker characterized as genomic regions with different DNA methylation status across different biological samples (e.g., DNA from healthy versus diseased cell of tissue). DMRs may be any suitable length (e.g., spanning between 2-500 nucleotides, or spanning between 10-400 nucleotides, or spanning between 50-300 nucleotides, or spanning between 100-200 nucleotides). The DMRs may comprise contiguous methylated nucleobases, or some pattern of interspersed methylated nucleobases.

The term “methylome”, as used herein, refers to the amount or pattern of methylation at different sites or regions within a population of cells. Thus, methylome may refer to the methylation score for a particular population of cells. For example, a disease state may have a methylome, such as the BAV methylome versus BAV-associated aortopathy. A tissue type may have a methylome. A cellular phenotype may have a methylome. The methylome may correspond to all of the genome, a subset of the genome (e.g., repeat elements in the genome), or a portion of the subset (e.g., those areas found to be associated with disease).

Other examples of methylomes include the methylomes of organs (e.g. methylomes of the liver, lungs, prostate, gastrointestinal tract, bladder etc.) that can contribute DNA into a bodily fluid (e.g. plasma, serum, sweat, saliva, urine, genital secretions, semen, stools fluid, diarrhea) fluid, cerebrospinal fluid, secretions of the gastrointestinal tract, ascitic fluid, pleural fluid, intraocular fluid, fluid from a hydrocele (e.g. of the testis), fluid from a cyst, pancreatic secretions, intestinal secretions, sputum, tears, aspiration fluids from breast and thyroid, etc.). The organs may be transplanted organs.

A methylome from plasma may be referred to a “plasma methylome”. The plasma methylome is an example of a cell-free methylome since plasma and serum include cell-free DNA (cfDNA).

The term “hypomethylation”, as used herein, refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypermethylation”, as used herein, refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “cfDNA methylome”, as used herein, refers to the pattern of methylation occurring in the genome of a cell, and includes any DMR, if present.

The term “detection”, as used herein, may refer to any process of observing a marker, or a change in a marker (such as for example the change in the methylation state of the marker), in a biological sample, whether or not the marker or the change in the marker is actually detected. In other words, the act of probing a sample for a marker or a change in the marker, is a “detection” even if the marker is determined to be not present or below the level of sensitivity. Detection may be a quantitative, semi-quantitative or non-quantitative observation

The “level” of one or more biomarkers means the absolute or relative amount or concentration of the biomarker in the sample. The term “level” also refers to the absolute or relative amount of methylation of the biomarker in the sample.

The terms “measuring” or “measurement,” or alternatively “detecting” or “detection,”, as used herein, refers to assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The term “methylation assay”, as used herein, refers to any assay for determining the methylation state of one or more CpG dinucleotide sequences within a sequence of DNA.

The terms “methylation state” or “methylation status”, as used herein, refers to the presence or absence of 5-methylcytosine (“5-mCyt”) at one or a plurality of CpG dinucleotides within a DNA sequence. Methylation states at one or more particular CpG methylation sites (each having two antiparallel CpG dinucleotide sequences) within a DNA sequence include “unmethylated,” “fully-methylated” and “hemi-methylated.”

A “reference level” or “control level” or “comparator control” of, for example, a methylome refers to a level of the methylome, for example level of methylation of the methylome that is indicative of a particular disease state, phenotype, or lack thereof, as well as combinations of disease states, phenotypes, or lack thereof.

A “positive” reference level refers to a level that is indicative of a particular disease state or phenotype. A “negative” reference level refers to a level that is indicative of a lack of a particular disease state or phenotype.

The term “risk of developing”, as used herein, refers to a probability that an individual will develop to a definitive diagnosis of disease. Determining probability includes both precise and relative probabilities such as “more likely than not”, “highly likely”, “unlikely”, or a percent chance, e.g., “90%”.

Risk can be compared with the general population or with a population matched with the subject based on any of age, sex, genetic risk, and environmental risk factors. In such a case, a subject can be determined to be at increased or decreased risk compared with other members of the population.

As used herein, the term “diagnosis” refers to a classification of an individual as having or not having a particular pathogenic condition, including, e.g BAV-associated aortopathy.

As used herein, the term “prognosis” refers to the predicted course, e.g., the likelihood of progression, of the condition. For example, a prognosis may include a prediction that severity of the condition is likely to increase, decrease or remain the same at some future point in time. In the context of the present disclosure, prognosis can refer to the likelihood that an individual: (1) will develop BAV-associated aortopathy or (2) has BAV-associated aortopathy.

As used herein, the term “progression” refers to a change, or lack thereof, in stage or severity of a condition over time. This includes an increase, a decrease or stasis in severity of the condition. In certain embodiments, rates of progression, that is, change over time, are measured.

The “sample” as used herein refers to a sample, typically derived from a biological fluid, cell, tissue, organ, or organism, comprising a nucleic acid or a mixture of nucleic acids comprising at least one nucleic acid sequence. Such samples include, but are not limited to sputum/oral fluid, amniotic fluid, blood, a blood fraction, or fine needle biopsy samples (e.g., surgical biopsy, fine needle biopsy, etc.) urine, peritoneal fluid, pleural fluid, and the like.

In a specific example, the sample is a blood sample.

In another example, the sample is a plasma sample.

A sample may processed to remove cells in order to produce a cell-free sample (e.g., cell-free plasma or serum). In some examples, cells may be removed from a sample via centrifugation, chromatography, electrophoresis, or any other suitable method.

In some examples, the biological sample (e.g., blood or plasma) is treated or processed by known methods to obtain the cell-free DNA present therein.

DNA methylation occurs at CpG sites across the genome and regulates gene expression. Cytosine is one of a group of four building blocks (i.e., nucleotides) from which DNA is constructed (i.e. cytosine (C), thiamine (T), adenine (A), and guanosine (G)). The chemical structure of cytosine is in the form of a six-sided hexagon or pyrimidine ring. Cytosine can be paired with guanosine in a linear sequence along the single DNA strand to form 5′-CG-3′, or CpG pairs. “CpG” refers to a cytosine-phosphate-guanosine chemical bond in which the phosphate binds the two nucleotides

Methylation may be measured as will be known to the skilled worker.

In some examples, bisulfite sequencing is used to determine and/or detect DNA methylation in a DNA sample (e.g., to identify a DMR).

In some examples, methylation specific PCR utilizes bisulfite treatment of a nucleic acid to detect methylation. For a base sequence modified by bisulfite treatment, PCR primers corresponding to regions in which a 5′-CpG-3′ base sequence is present may be constructed. For example, two kinds of primers corresponding to the methylated case and the unmethylated case may be generated. More specifically, primer pairs may thus be designed to be “methylated-specific” by including sequences complementing only unconverted 5-methylcytosines, or, on the converse, “unmethylated-specific”, complementing thymines converted from unmethylated cytosines. Methylation is determined by the ability of the specific primer to achieve amplification. When genomic DNA is modified with bisulfite and then subjected to PCR using the two kinds of primers, if DNA is methylated, then a PCR product can be made from the DNA from a primer corresponding to the methylated base sequence. In contrast, if that region of the gene is unmethylated, a PCR product can be made from the DNA based on a primer corresponding to the unmethylated base sequence. The methylation of DNA can be qualitatively analyzed, e.g., using agarose gel electrophoresis.

In some examples, real-time methylation-specific PCR may be used and involve a real-time measurement method, such as real-time PCR, modified from methylation-specific PCR. The method may involve treating genomic DNA with bisulfite, and utilizing methylated-specific and unmethylated-specific PCR primers in combination with real-time PCR. In some examples, digital droplet PCR may be used.

In other examples, DNA sequencing, including single molecule sequencing, such as pyrosequencing or sequencing by ligation (e.g., SOLiD™), may be used to detect the presence, absence, or amount of methylation.

In other examples, base-specific cleavage/MALDI-TOF may be used to detect methylation

In other examples, methylation-sensitive single-strand conformation analysis (MS-SSCA) may be used to detect methylation.

In other examples, high-resolution melting analysis (HRM), a real-time PCR-based technique, may be used to detect methylation,

In other examples, contacting a nucleic acid sample with a methylation sensitive restriction endonuclease that cleaves only unmethylated CpG sites under conditions and for a time to allow cleavage of unmethylated nucleic acid may be used to detect methylation,

The term “treatment”, “treat”, or “treating” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

The term “amelioration” or “ameliorates” as used herein refers to a decrease, reduction or elimination of a condition, disease, disorder, or phenotype, including an abnormality or symptom.

The term “symptom” of a disease or disorder is any morbid phenomenon or departure from the normal in structure, function, or sensation, experienced by a subject and indicative of disease.

In some examples, “treatment” refers to the care given to a subject in order to reduce, eliminate, and/or prevent the severity and/or frequency of BAV-associated aortopathy symptoms, and to improve or remediate the damage caused by BAV-associated aortopathy.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

Abstract

Background: The dilation of the aorta that occurs as a consequence of a congenitally bicuspid aortic valve (BAV) is associated with a risk of dissection, aneurysm or rupture. With progressive aortopathy, surgery is often recommended but current patient selection strategies have limitations. A blood-based assay to identify those who would most benefit from prophylactic surgery would be an important medical advance. In a proof-of-concept study we sought to identify aorta-specific differentially methylated regions (DMRs) detectable in plasma cell-free DNA (cfDNA) obtained from patients undergoing surgery for BAV-associated aortopathy.

Methods: We used bioinformatics and publicly-available human methylomes to identify aorta-specific DMRs. We used data from 4D-flow cardiac magnetic resonance imaging to identify regions of elevated aortic wall shear stress (WSS) in patients with BAV-associated aortopathy undergoing surgery and correlated WSS regions with aortic tissue cell death assessed using TUNEL staining. Cell-free DNA was isolated from patient plasma and levels of candidate DMRs were correlated with aortic diameter and aortic wall cell death.

Results: Aortic wall cell death was not associated with maximal aortic diameter but was significantly associated with elevated WSS. We identified 24 candidate aorta-specific DMRs and selected 4 for further study. A DMR on chromosome 11 was specific for the aorta and correlated significantly with aortic wall cell death. Plasma levels of total and aorta-specific cfDNA did not correlate with aortic diameter.

Conclusions: In a cohort of patients undergoing surgery for BAV-associated aortopathy, elevated WSS created by abnormal flow hemodynamics was associated with increased aortic wall cell death which supports the use of aorta-specific cfDNA as a potential tool to identify aortopathy and stratify patient risk.

Background

The aortopathy that occurs as a consequence of a congenitally bicuspid aortic valve (BAV) is associated with a risk of dissection, aneurysm or rupture (1). The etiology of BAV-associated aortopathy appears to be multifactorial and related to inherent genetic defects (2) combined with hemodynamic wall shear stress (WSS) created by turbulence across the abnormal valve (3-6). Increased aortic WSS leads to the degradation of elastin and the dysregulation of extracellular matrix proteins which are linked to smooth muscle cell death (7-9). With progressive aortopathy, aortic valve replacement surgery is often recommended but patient selection is controversial and often dependent upon individual and institutional practice (10-14). A blood-based assay, potentially in combination with advanced imaging techniques, to identify those who would most benefit from prophylactic surgery would be a significant advance (15).

Cell-free DNA (cfDNA) refers to fragments of genomic DNA released into the blood during cellular apoptosis (16-18). The accessibility of plasma cfDNA and its retention of genetic and epigenetic changes has resulted in the development of cfDNA-based diagnostic assays for diverse human diseases and applications (19-21). DNA methylation is an important regulator of gene expression and determinant of cell specialization (22) and tissue-specific differentially methylated regions (DMRs) have been identified for multiple human cells, tissues and organs (20). We hypothesize that aorta-specific DMRs detectable in cfDNA will allow the non-invasive assessment of disease, the prediction of important clinical events and enable precision medicine for optimal management of patients with aortopathy and highly variable individual risk.

In this pilot study we identified novel and unique aorta-specific DMRs that could be measured in human plasma cfDNA obtained from patients with BAV-associated aortopathy who were undergoing surgery. We identified the relationship between elevated WSS and cell death in the ascending aorta of human BAV patients to demonstrate the biological rationale for cfDNA as a biomarker of aortopathy and identified an association between aorta-specific cfDNA levels, WSS and aortic wall cell death.

Results

Cell Death is Increased in Aortic Regions of Elevated Wall Shear Stress

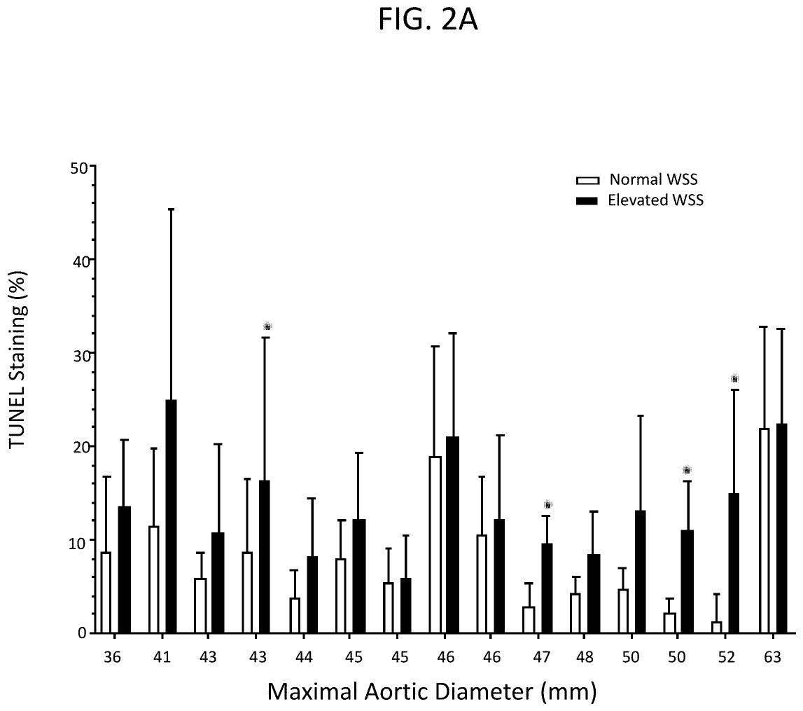

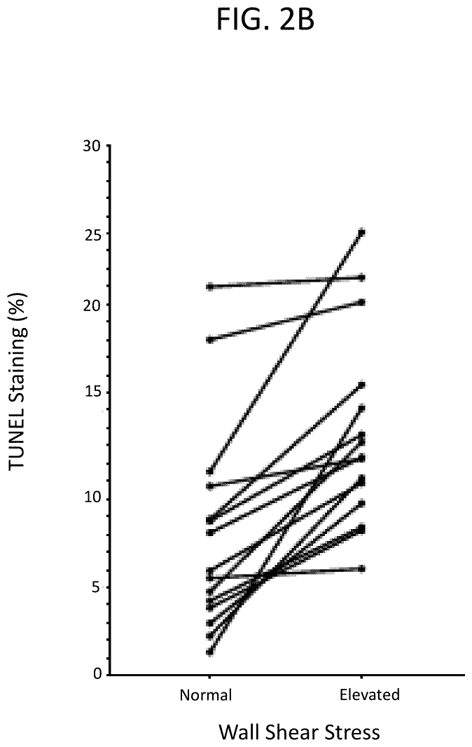

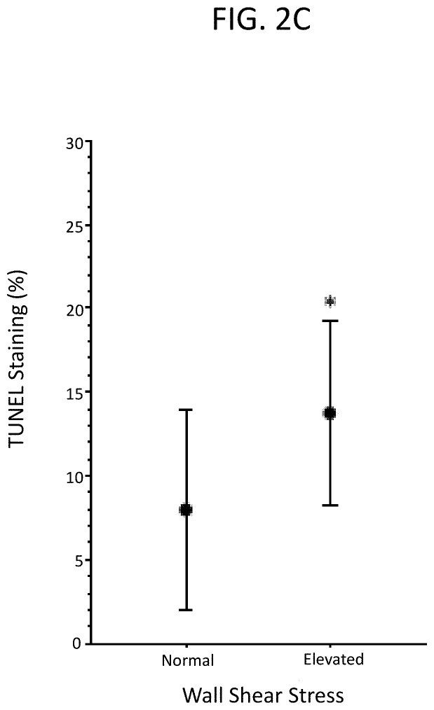

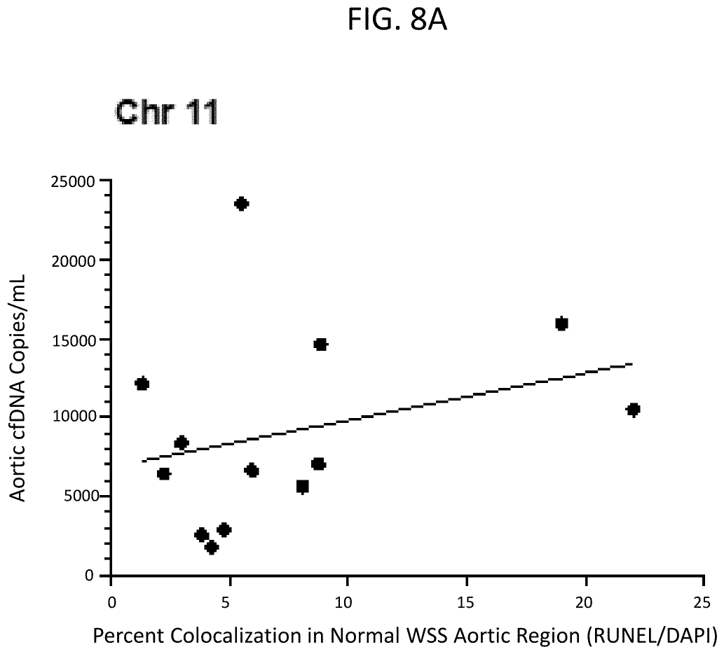

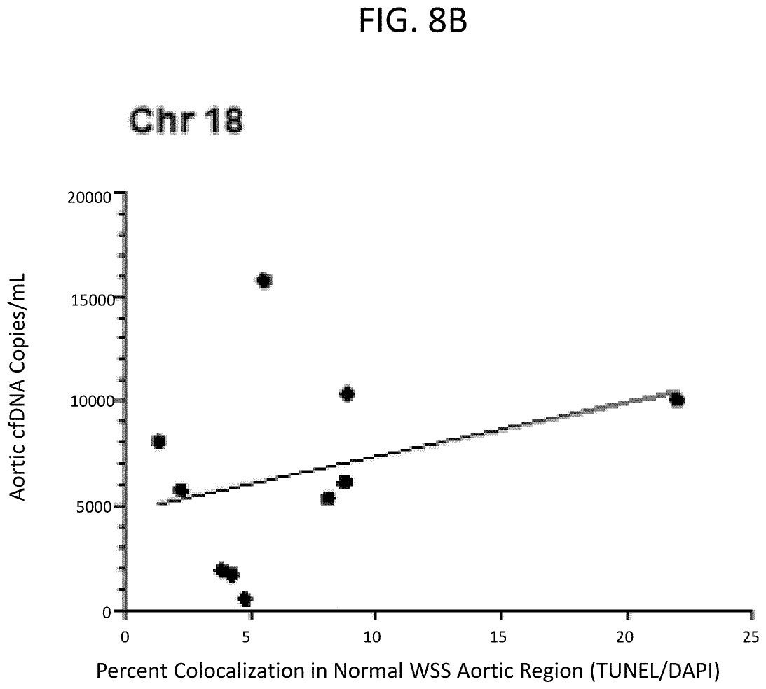

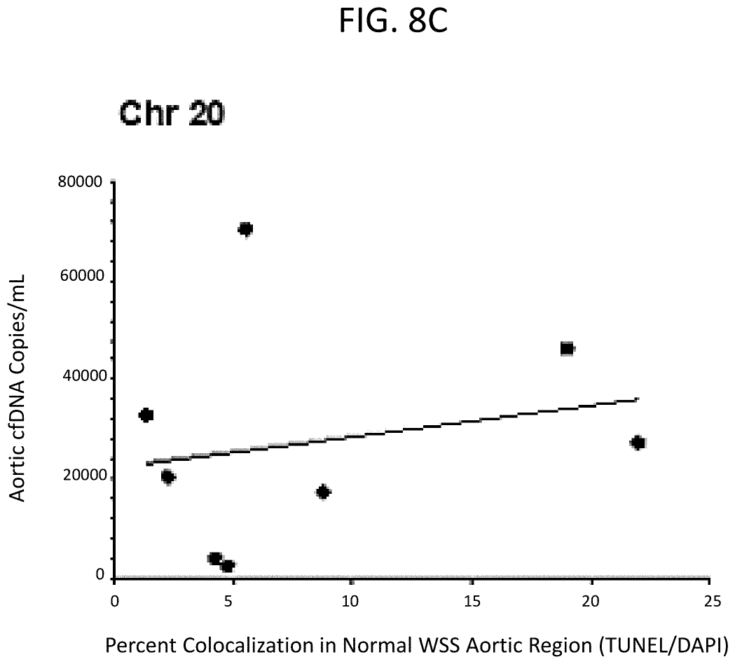

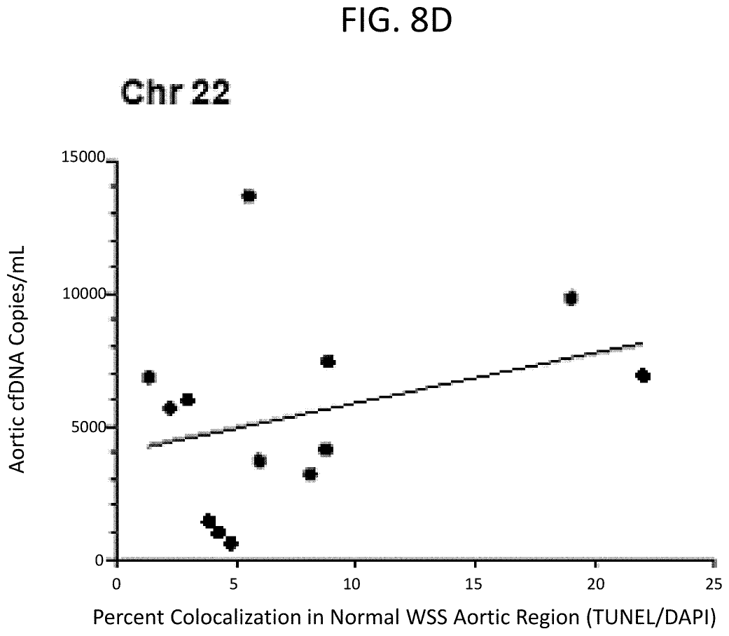

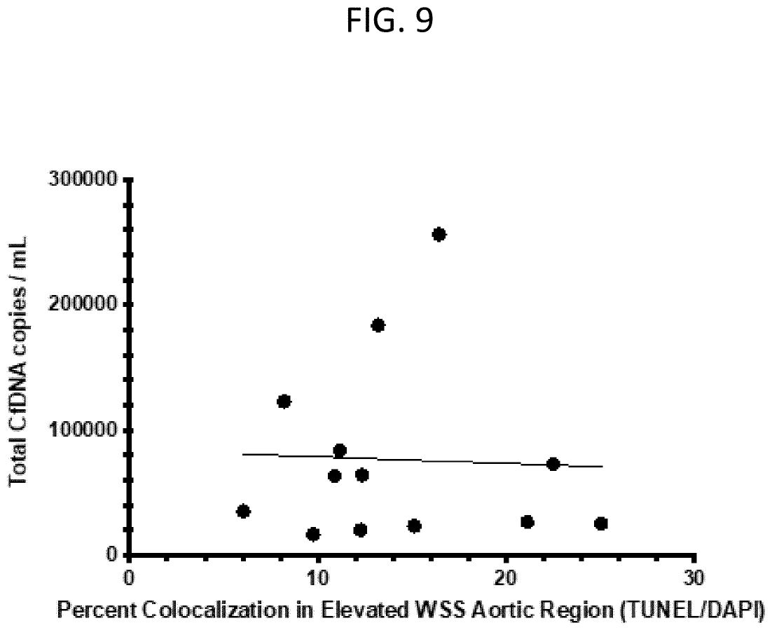

For all individual BAV patients, cell death, as assessed using terminal deoxynucleotidyl transferase dUTP nick end labeling (TUNEL) and 4′,6-diamidino phenylindole (DAPI) staining, was increased in regions of the ascending aorta that demonstrated elevated WSS compared to the regions of normal WSS as determined by cardiac magnetic resonance imaging (CMR) and this difference was significant for the paired comparisons and pooled data (FIG. 2). The mean cell death (mean percent colocalization of TUNEL and DAPI staining) in the regions with normal WSS was 7.98±5.94% compared to 13.74±5.49% in regions with elevated aortic WSS (P=0.0027). This data demonstrates that regions of abnormal hemodynamics are associated with increased levels of aortic tissue cell death. However, we did not observe a consistent relationship between maximal aortic diameter and WSS or cell death. There was no significant relationship found between levels of aorta-specific cfDNA and cell death in regions with normal WSS (FIGS. 8A-D) and total cfDNA did not correlate with levels of cell death in regions of increased WSS (FIG. 9).

FIG. 2 depicts A, Aortic wall cell death (indicated by percentage of TUNEL-positive cells) for 15 patients with varying maximal aortic diameters comparing regions of normal and elevated aortic WSS. Data presented are mean±SD with * indicating p<0.05 as determined by a Mann-Whitney U test. B, Paired patient data was found to be significantly different (p=0.00006) using the Wilcoxon signed-rank test. C, Summarized data. Data are mean±SD with significantly greater cell death in regions of elevated WSS as determined by a Mann-Whitney U test (p=0.0027).

Identification of Aorta-Specific Differentially Methylated Regions

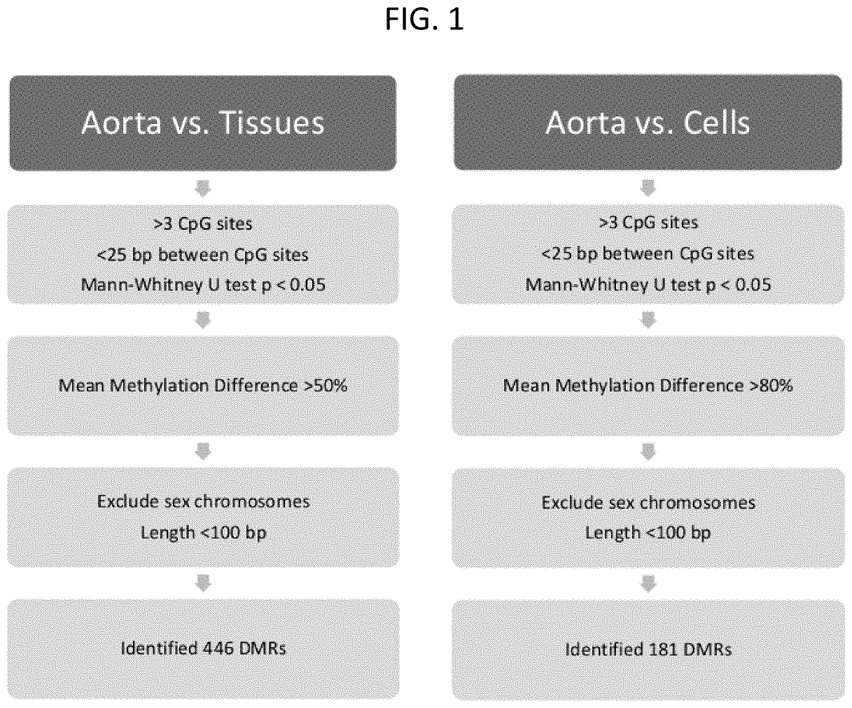

After comparing the aortic methylome to methylomes from 19 tissues we identified 446 putative aortic-specific DMRs with a minimum mean methylation difference of 60%. Comparing the aorta methylome to 14 methylomes from various hematopoietic cells identified 181 aorta-specific DMRs with a minimum mean methylation difference of 90% (FIG. 1). There were 24 DMRs on autosomal chromosomes that were common to both datasets and after further testing we selected four DMRs for further study, all of which were hypomethylated in the aorta (Table 2). The DMR on Chr 11 (position Chr 11:3,168,734-3,168,832) is 73 bp in length, contains 6 CpG sites and is predicted in silico to have the lowest specificity for the aorta in comparison to other tissues but the highest specificity in comparison to hematopoietic cells. The DMR on Chr 18 (position Chr 18:74,171,459-74,171,505) contains 8 CpG sites over 46 bp. The DMR on Chr 20 (position Chr 20:45,860,400-45,860,466) contains 6 CpG sites over 65 bp with the greatest predicted specificity for the aorta in comparison to other tissues. The DMR on Chr 22 (position Chr 22:27,999,645-27,999,717) contains 7 CpG sites over 72 bp. Given the unbiased nature of our enquiry, we were not surprised to see that the regions identified were not specific to genes known to be associated with cardiovascular disease or development. The Chr 11 DMR is found within the OSBPL5 (oxysterol binding protein-like 5) gene which is an intracellular lipid receptor. The Chr 18 DMR is found within the ZNF516 (zinc finger protein 516) gene, the Chr 20 DMR is within the ZMYND8 (zinc finger, MYND-type containing 8) gene and the Chr 22 DMR is not associated with a coding region. A review of these four regions using the ENCODE database did not identify obvious regulatory functions.

| [0096] | Table 2 Four candidate aorta-specific DMRs | | | | |
|---|---|---|---|---|---|
| Chr | DMR | Length | CpGs | Mean Methylation | Difference (%) |
| | Start Position | | | Non-aortic tissues | Hematopoietic |
| | | | | | cells |
| 11 | 3,168,734 | 73 | 6 | 70.60 | 92.75 |
| 18 | 74,171,459 | 46 | 8 | 74.26 | 91.27 |
| 20 | 45,860,400 | 65 | 6 | 86.88 | 81.01 |
| 22 | 27,999,645 | 72 | 7 | 75.36 | 91.28 |

Data presented for the four DMRs studied include chromosomal location, length (in base pairs), number of CpG sites and methylation differences between the aorta and pooled non-aortic tissues and hematopoietic cells as determined computationally. All DMRs presented are hypomethylated in the aorta compared to non-aortic tissues and hematopoietic cells. Chr, chromosome; CpG, cytosine-guanine nucleotides.

In Vitro Testing of Aorta-Specific DMRs

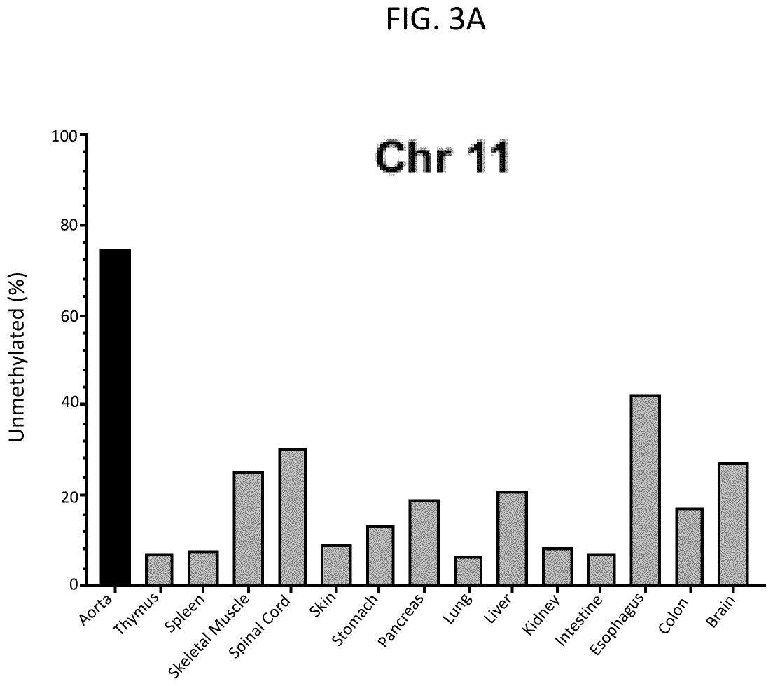

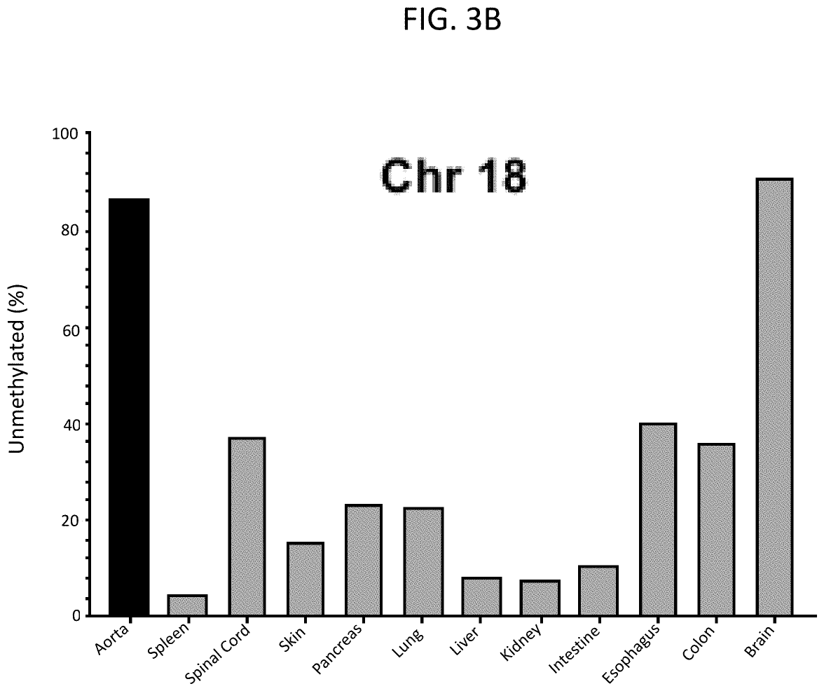

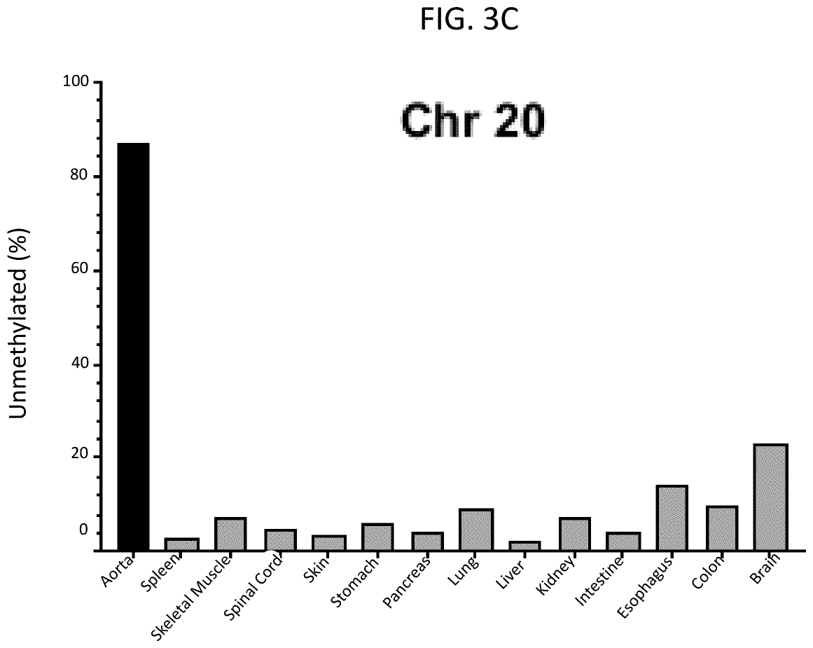

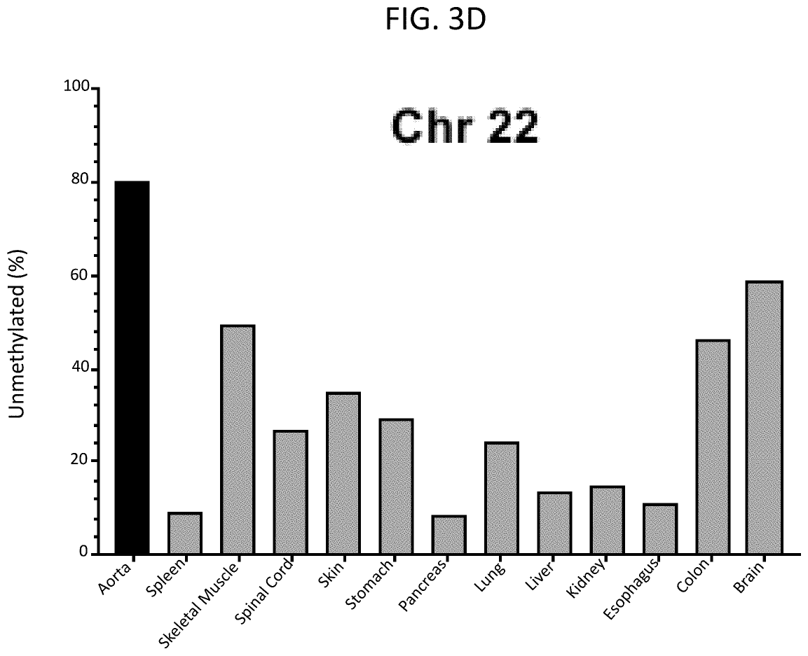

To further test the tissue-specificity of the DMRs that we had identified, we obtained gDNA from 14 different human tissues and organs encompassing all three developmental germ layers and compared the measured methylation status in each tissue to the aorta for each DMR. For the Chr 11 DMR, 74.2% of the reads from the aorta were unmethylated, for the Chr 18 DMR 86.8% of the reads were unmethylated, 86.8% of the reads were unmethylated for the Chr 20 DMR and for the Chr 22 DMR, 80.4% of the aorta reads were unmethylated (FIG. 3). The Chr 11 and Chr 20 DMRs were mostly methylated across all tissues but the Chr 18 DMR was mostly unmethylated in the brain with levels similar to that seen in the aorta and also showed low levels of methylation in the spinal cord, esophagus and colon. For the Chr 22 DMR it was highly unmethylated in skeletal muscle, colon and brain.

Correlation of Aorta-Specific cfDNA Levels with Clinical Severity of Aortopathy

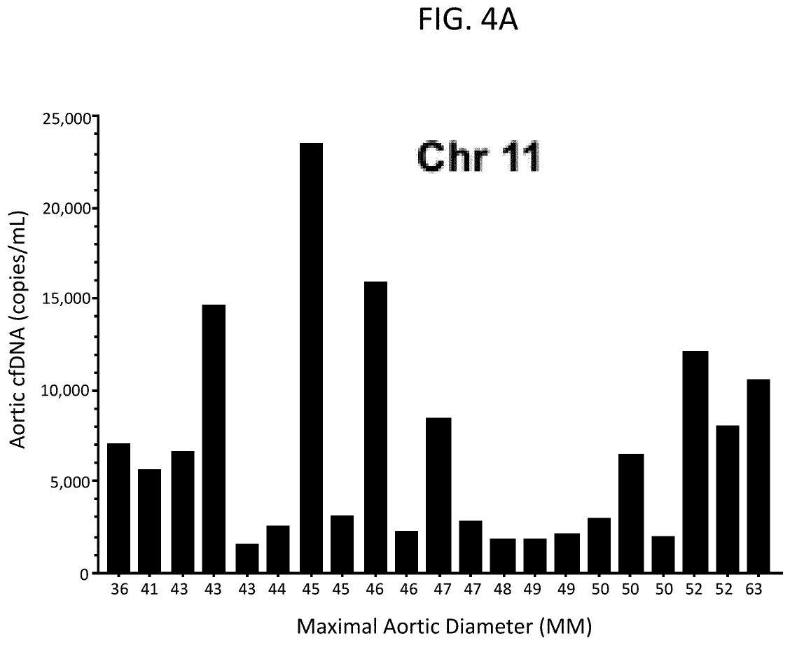

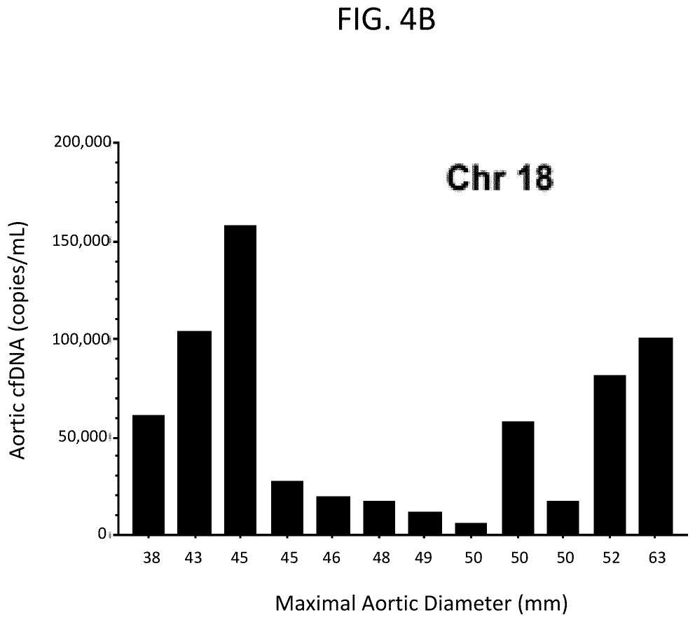

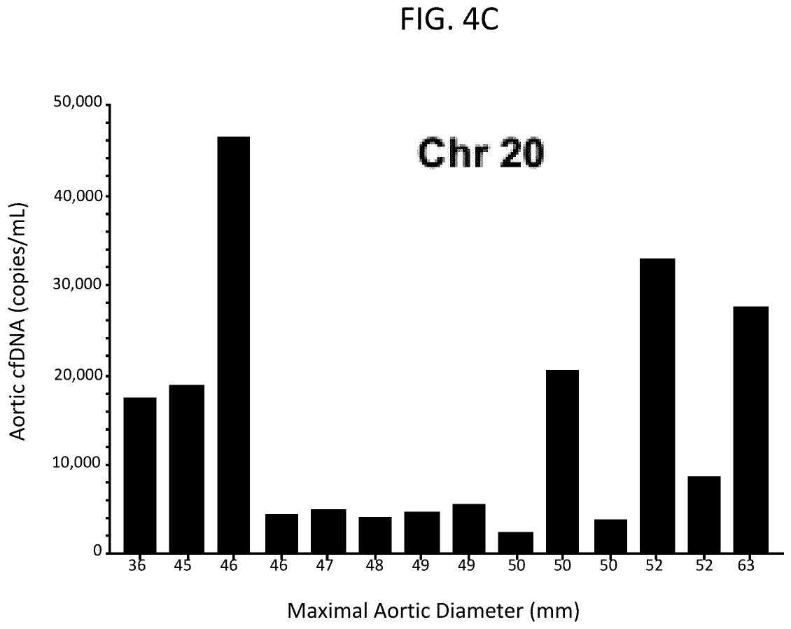

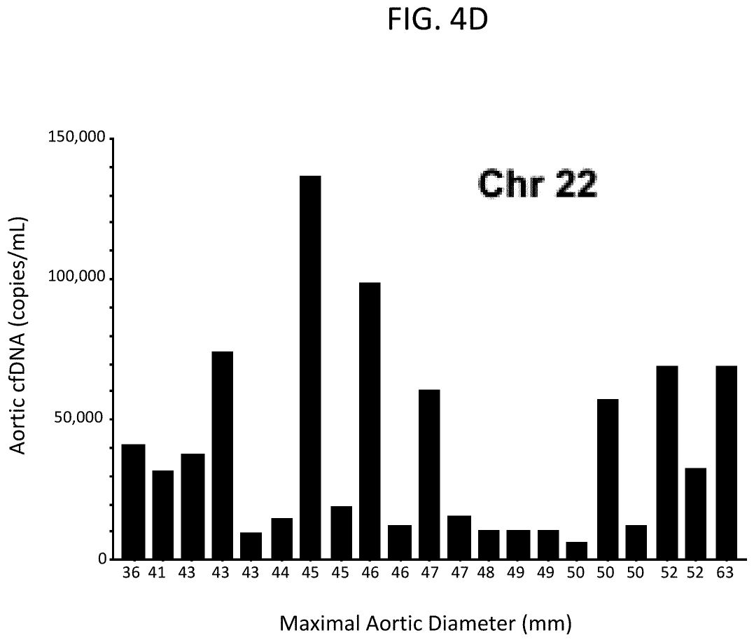

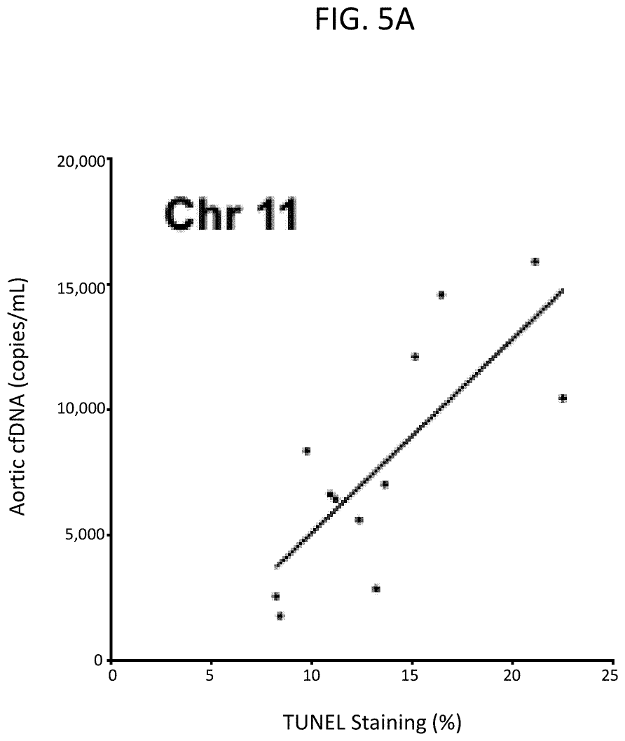

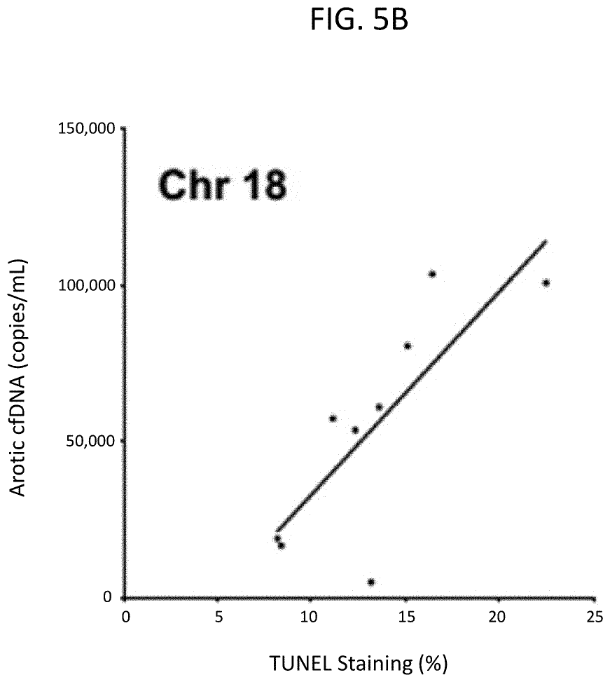

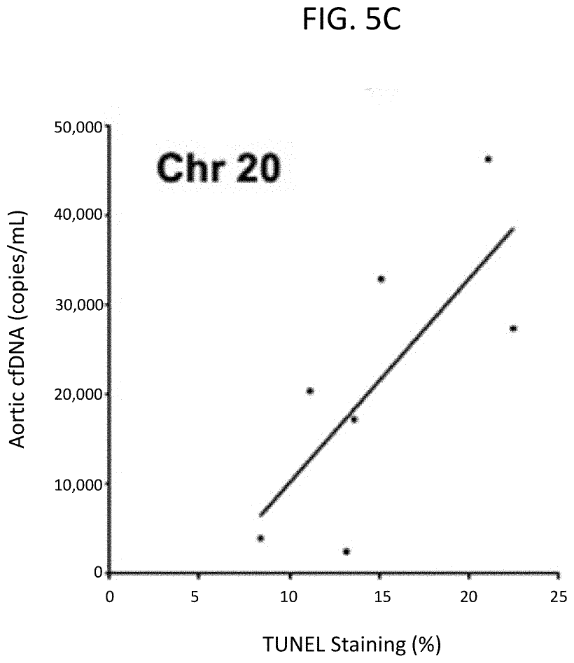

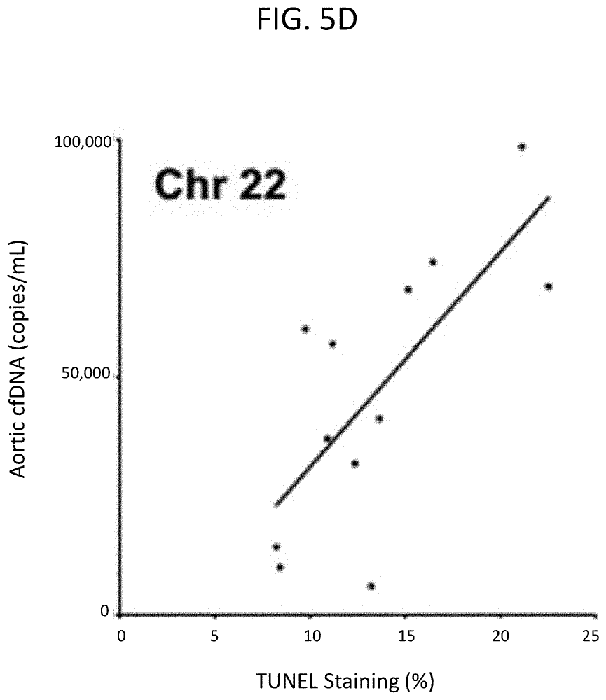

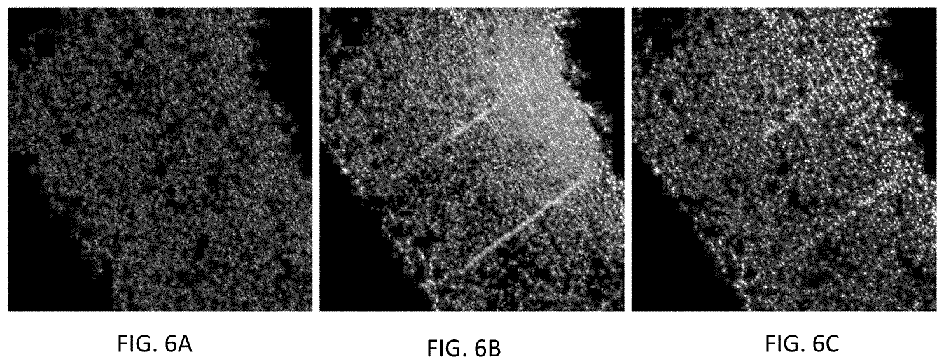

Using cfDNA isolated from patient plasma, the aorta-specific cfDNA levels for 23 BAV patients were determined using the DMRs located on Chr 11, 18, 20 and 22 (FIG. 4). In this cohort of patients with severe disease, none of the aorta-specific DMRs showed an association with aortic diameter as measured using CMR. Similarly, total plasma cfDNA also did not correlate with aortic size (FIG. 6). However, levels of aorta-specific cfDNA as determined based on our 4 DMRs did show a positive correlation with levels of cell death in the regions of elevated aortic WSS (FIG. 5). This correlation was statistically significant for the Chr 11 DMR (R²=0.59, p=0.0035), the Chr 18 DMR (R²=0.62, p=0.012) and the Chr 22 DMR (R²=0.52, p=0.0078). The Chr 20 DMR did not show a significant correlation (R²=0.55, p=0.06). However, our candidate DMRs showed no significant correlation with several histological markers associated with elastin degradation (Table 6) and dysregulation of ECM proteins (concentrations of MMP type 1, 2 and 3, TGFβ-1 and TIMP-1, Table 7) known to occur in aortopathy. Due to low plasma volumes and resulting low yields of cfDNA, some patient cfDNA samples could not be sequenced which affected the number of data points for each DMR as displayed in FIGS. 4 and 5.

Discussion

Using publicly-available human methylomes we bioinformatically identified 24 candidate aorta-specific DMRs. A subset were chosen for further evaluation and the Chr 11 DMR demonstrated specificity for the aorta and significant correlation with aortic wall apoptosis. Our results in this proof-of-concept study demonstrate the feasibility of identifying a peripheral blood marker for aortopathy based on DNA methylation patterns and the importance of in vitro validation studies.

In this study, the levels of cell death were measured using the TUNEL assay and colocalized to individual cells using nuclear DAPI staining in aortic tissue samples from regions of elevated and normal WSS in BAV patients requiring surgery. We found that cell death was not associated with maximal aortic diameter but was associated with increased WSS. Our data suggests an association between abnormal hemodynamics and ongoing tissue injury that is independent of aortic size. As these BAV patients experience chronic localized elevated WSS, we hypothesize that the greater levels of cell death in these regions would lead to the depletion of vascular smooth muscle cells and likely negatively impact the integrity of the aorta. Our subsequent data suggests that this increased cell death due to hemodynamic stress is detectable by circulating aorta-specific cfDNA identified through unique DNA methylation patterns.

DNA methylation plays a critical role in regulating gene expression and thus cellular differentiation. Tissue-specific methylation patterns are conserved within a tissue type and to a large degree across individuals (23). This consistency is critical for the potential development of a universal, minimally-invasive cfDNA-based assay. From the 24 putative aorta-specific DMRs identified, 4 were selected for detailed study based in part on their computationally-predicted specificity for the aorta. The specificity of the 4 DMRs on Chr 11, 18, 20 and 22 was then tested in vitro using a panel of DNA isolated from multiple human tissues. We found that the methylation differences between the aorta and several tissues and organs were smaller than the in silico predictions for many of the DMRs. Importantly, the DMR on Chr 18 was found to be equally hypomethylated in the brain and the aorta and therefore can be eliminated as an aorta-specific biomarker.

Our candidate DMRs were also evaluated using plasma cfDNA obtained from patients with BAV-associated aortopathy undergoing surgery. The levels of both aorta-specific and total plasma cfDNA did not correlate with maximal aortic diameter which is consistent with numerous studies documenting that aortic dimensions (both absolute diameter and rate of progression) are insufficient for assessing the severity of aortopathy and in estimating the underlying risk for aortic rupture (12, 24-26). However, although our aorta-specific DMRs did not correlate with aortic dimension, three of them did correlate significantly with levels of cell death in the aorta. This encouraging result provides a rationale for cfDNA as a biomarker for aortic cell death which, as we have also shown, is associated with elevated regions of WSS. Although our candidate DMRs showed no significant correlation with several histological markers associated with elastin degradation and dysregulation of ECM proteins known to occur in aortopathy, the integrity of the aortic wall is impacted by many factors, including apoptosis that leads to the depletion of vascular smooth muscle cells (27-29). Regardless of the mechanism of cell death, DNA fragmentation and its release into the circulation is a common endpoint. Thus, the level of aorta-specific cfDNA is potentially an independent and end-stage measure of aortic cell death, regardless of mechanism.

Conclusions

In conclusion, elevated WSS created by abnormal flow hemodynamics is associated with increased aortic wall cell death. This finding supports the use of cfDNA as a tool to identify aortopathy and stratify patient risk. By leveraging publicly-available data and developing a novel bioinformatics pipeline we identified candidate aorta-specific DMRs that were detectable in cfDNA. Subsequent in vitro studies suggest that the Chr 11 DMR is specific for the aorta and we have shown that it also correlated with the severity of aortic wall apoptosis in patients with severe aortopathy requiring surgery.

Methods

Patient Data

This study was approved by the Research Ethics Boards at the University of Calgary and Northwestern University. After obtaining informed written consent, blood and matched tissue samples from adult patients with BAV undergoing aortic surgery were obtained (Table 1). All patients (n=23) and healthy age-matched controls with tricuspid aortic valves and no documented cardiovascular disease (n=10) underwent 4D-flow CMR to visualize aortic blood flow patterns, generate WSS heat maps and calculate WSS values as previously described (9). Data from the healthy controls (who did not undergo surgery) were used to construct a physiologically normal heat map of aortic flow and regions of depressed, normal and elevated WSS were determined for each BAV patient relative to this control map. Aortic wall tissue samples collected during surgery were flash frozen and then paraffin-embedded.

[00112]Table 1 Patient clinical characteristics

| Parameter | Study Population |
|---|---|
| Age (years) | 49.3 ± 14.9 |
| Male (%) | 20 (87) |
| BAV Classification: | |
| Type 0 (lateral) | 1 |
| Type 1 (RL) | 15 |
| Type 2 (RN) | 7 |
| Maximum aortic diameter (cm) | 4.67 ±0.52 |
| Aortic Valve Surgical Procedure: | |
| Repair | 3 |
| Replacement | 20 |
| Ascending Aorta Surgical Procedure: | |
| Ascending aorta replacement | 20 |
| Root replacement | 2 |
| Hemi-arch reconstruction | 1 |

Data reported as n or mean with standard deviation. BAV types were grouped according to the Sievers classification scheme (36). RL, right-left coronary cusp fusion; RN, right-non coronary cusp fusion.

Isolation of cfDNA

Adult BAV patients were recruited and 8-10 mL of blood was collected before the CMR imaging required for their surgery. The blood was centrifuged at 1900×g for 10 minutes and then the isolated plasma was centrifuged at 4° C. at 13,000 RPM for 15 minutes. The resulting plasma was stored at −80° C. until use. Patient cfDNA was isolated from 2 mL of plasma using the semi-automated MagNA Pure 24 System (Roche) according to the manufacturer's instructions. The cfDNA yield was quantified by TapeStation (Agilent) and stored at −80° C.

TUNEL Assay for Cell Death

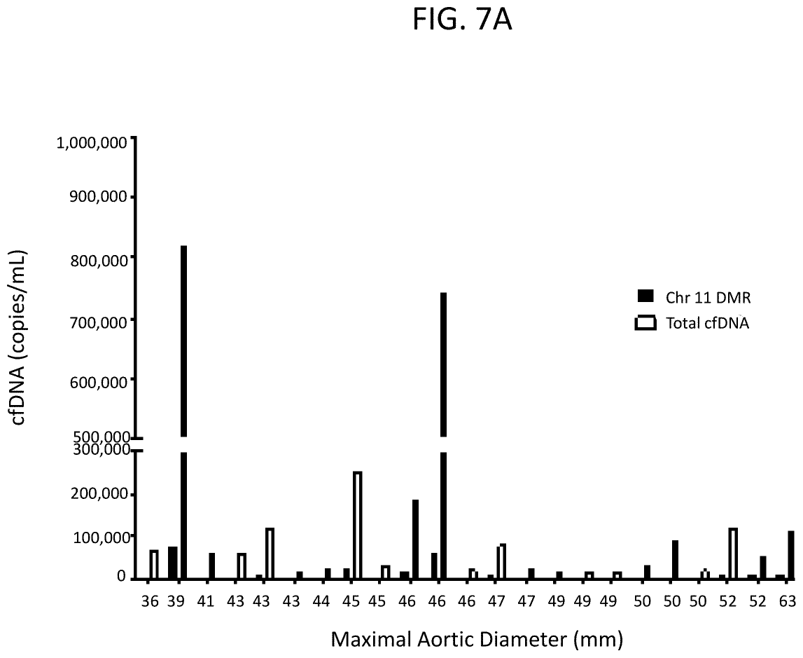

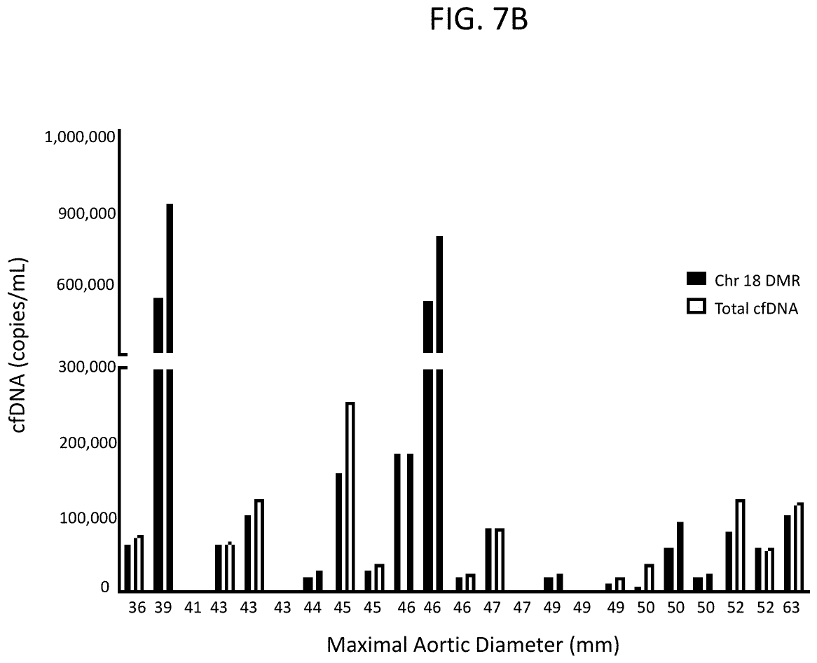

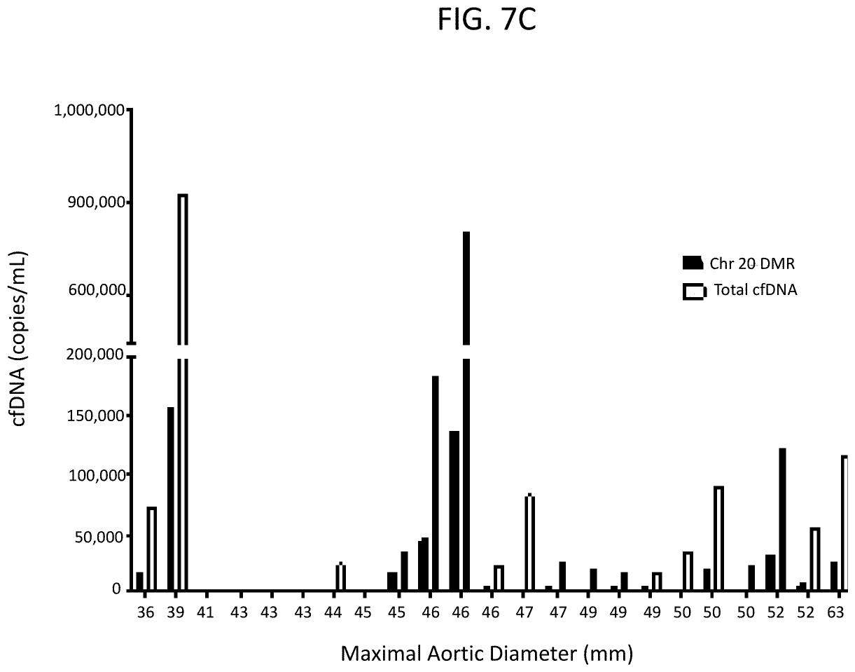

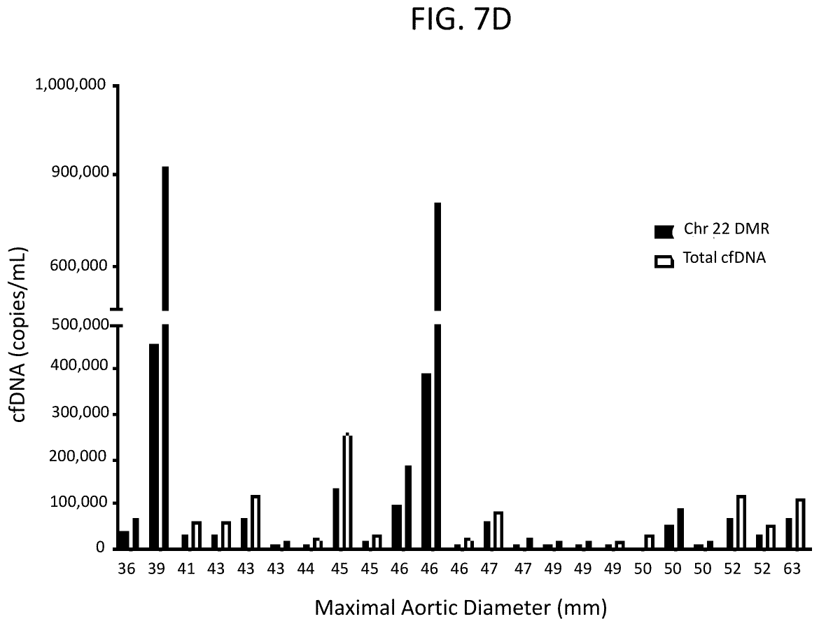

Regions of the ascending aorta with the highest and lowest WSS scores (one of each) were stained for quantification of cell death. Paraffin tissue sections mounted on glass slides were used for terminal deoxynucleotidyl transferase dUTP nick end labeling (TUNEL, Promega) and 4′,6-diamidino-2-phenylindole (DAPI, ThermoFisher Scientific) staining according to the manufacturer's instructions. Stained tissue slides were imaged (10-20 images per sample to capture the entire tissue section) using a spinning disk confocal super resolution microscope (SpinSR10, Olympus) at 10× magnification and then analyzed using the ImageJ plugin Fiji.(30) Green (488 nm) TUNEL staining was colocalized to blue (405 nm) DAPI staining (FIGS. 7A-D) and the percent colocalization/cell death was determined for each image. Detailed experimental details are described herein.

Identification of Aorta-Specific DMRs

Publicly-available methylomes from human hematopoietic cells (n=14), non-aortic tissues (n=21) and aorta (n=1) were obtained from the Roadmap and Blueprint Epigenomic projects (Table 3) (31, 32). The software package Metilene identified DNA regions that were significantly differentially methylated (mean difference >10%) based on a Mann-Whitney U test (alpha=0.05) (33). Other parameters included a minimum of 4 CpG sites per DMR, <25 base pairs (bp) separating each of the CpG sites and a total DMR length of <100 by to enable detection in fragments of cfDNA which are ˜140-160 bp in length. A second filtering step identified aortic DMRs with a mean methylation difference >60% for non-aortic tissues and >80% for hematopoietic cells. Comparisons were made separately to enable stringent exclusion of DMRs in peripheral lymphocytes which produce the majority of cfDNA in the circulation. The filtering cut-offs chosen resulted in a sufficient number of DMRs within each interrogated group to enable the identification of DMRs that were common to both groups. Shared DMRs on autosomal chromosomes (sex chromosomes were excluded to avoid dosage effects) identified from the comparison of aorta and non-aorta tissue methylomes (n=446) and from aortic tissue compared to hematopoietic cell methylomes (n=181) provided a total of 24 candidate aorta-specific DMRs (FIG. 1 and Table 4). Specific details of the bioinformatics used are described herein

For 10 candidate DMRs with the highest aorta-specificity, sequences containing the DMR of interest and 100 bp upstream and downstream were taken from a methylome of the aorta (GSM983648) generated by the NIH Roadmap Epigenomics project. These sequences were used as input for MethPrimer(34) to generate primers for amplification of the DMRs from bisulfite-converted genomic DNA (gDNA). Primers did not contain CpG islands and contained at least two non-CpG cytosines. Total product size was constrained to <150 bp to enable amplification from cfDNA. Optimal annealing temperatures were determined for all DMRs. From these, four DMRs, on chromosomes (Chr) 11, 18, 20 and 22 were chosen for further study as these regions amplified readily with no off-target products (Table 2). Sequences and optimal annealing temperatures for the primers used to amplify the DMRs are presented in Table 5.

To confirm the specificity of our DMRs identified in silico, we used a commercial panel (Zyagen) of gDNA from human brain, colon, esophagus, small intestine, kidney, liver, lung, pancreas, stomach, skin, spinal cord, skeletal muscle, spleen and thymus to compare methylation levels for all 4 DMRs across tissues in comparison to aorta tissue. Patient cfDNA and panel gDNA was bisulfite converted using the Epitect Bisulfite Kit (Qiagen) with elution performed twice using 20 μL of buffer EB warmed to 56° C. to improve yields. Bisulfite-converted DNA was used for PCR amplification of the DMRs and products were visualized on a 3% Tris-Acetate-EDTA agarose gel and quantified by TapeStation (Agilent).

Next-Generation Sequencing

For quantification of methylation, PCR amplicons were pooled in equimolar concentrations for sequencing on an Illumina MiSeq. Libraries were sequenced using the MiSeq reagent kit v3 (150 cycles) to produce 2×75 bp paired-end reads. The quality of each sequenced pool was assessed using FastQC (35). The bisulfite sequencing plugin for the CLC Genomics Workbench (Qiagen) was utilized to analyze the reads which were mapped to the hg19 reference genome with a minimum acceptable alignment of >80%. Methylation levels were determined for each individual CpG site within each DMR and mean methylation levels were calculated.

Determination of cfDNA Concentrations

The concentration of cfDNA (ng/mL) extracted from 2 mL of patient plasma was divided by 0.00303 ng (mass of a single human genome) to determine the total concentration of cfDNA (copies/mL) in recipient plasma (20). To determine the absolute concentration of each DMR, the copies/mL was multiplied by the fraction of unmethylated molecules within the given pool of amplicons as determined from the analysis of the bisulfite-sequenced reads.

Statistics

Statistical analysis was performed using GraphPad Prism 8.0. The percentage of cell death in the aortic tissue is presented as a mean±standard deviation. The Shapiro-Wilk test was used to determine normality. For cell death, a paired comparison between regions of normal and elevated WSS was conducted for each patient using a Wilcoxon signed rank test and the comparison between pooled patients was performed using the Mann-Whitney test with p<0.05 considered to be significant. Linear regression to calculate R² was used to correlate the DMR-specific cfDNA levels with the percentage of aortic wall cell death as reported in Table 7.

List of Abbreviations

BAV: bicuspid aortic valve; cfDNA: cell-free DNA; Chr: chromosome; CMR: cardiac magnetic resonance imaging; CpG: cytosine-guanine nucleotides; DAPI: 4′,6-diamidino-2-phenylindole; DMR: differentially methylated region; gDNA: genomic DNA; MRI: magnetic resonance imaging; PCR: polymerase chain reaction; TUNEL: terminal deoxynucleotidyl transferase dUTP nick end labeling; WSS: wall shear stress.

Additional Materials

Analysis of Myocardial Apoptosis

A green fluorescent signal was observed at 488 nm in regions that contained apoptotic or necrotic myocardial cells while DAPI-stained nuclei emitted a blue signal at 405 nm. Images were analyzed using ImageJ and the Fiji plugin. The brightness and contract were adjusted to a minimum intensity of 122 arbitrary units (a.u.) and a maximum intensity of 145 a.u. for each image taken at 488 nm while all images taken at 405 nm were adjusted to a minimum intensity of 105 and a maximum intensity of 145. This allowed for the clearest visualization of the TUNEL and DAPI signals within each set of images as these settings removed any background staining within the images. Then, using the selection tool, the image of the tissue section taken at 488 nm was traced and edges and any areas of thin or torn tissue were omitted. This ensured that any cells that stained positively for TUNEL and DAPI due to physical damage, rather than immune-mediated damage, did not skew the results. This traced outline was then pasted and aligned with the image taken at 405 nm which contained all of the DAPI-stained cells. The traced outlined was applied, which removed all areas of the image outside of the outline, leaving behind only the tissue section of interest. Next, both images taken at 488 nm and 405 nm were further enhanced by removing any residual noise using the “despeckle” option in Fiji. Following this, the cropped image taken at 405 nm was then used to create a mask, which served as a reference for the number and location of each nucleated cells within the tissue section of interest when determining the colocalization of TUNEL and DAPI. Finally, the “coloc 2” test in Fiji was employed. Channel 1 was assigned as the image taken at 488 nm and displayed all TUNEL-positive cells and channel 2 was assigned as the image taken at 405 nm and displayed all DAPI-positive cells. The mask created from the image taken at 405 nm was also plugged in. The “coloc 2” test was allowed to run and output listed the proportion of cells that displayed a colocalization of TUNEL and DAPI and, therefore, represented apoptotic or necrotic cells within the tissue section.

In Silico Identification of Candidate Ventricle-Specific DMRs

Converting File Formats

Big Wig to Wig: $ bigWigToWig<file_name.bw><file_name.wig>

Wig to Bed: $ convert2bed-i wig<file_name.wig><file_name.bed>

Bed to Bedgraph: $ awk ‘{print $1″\t″$2″\t″$3″\t″$5}’ file_name.bed>file_name.bedgraph

Sorted Bedgraph: $ sortBed-i file_name.bedgraph>file_name_sorted.bedgraph

Creating Input Files for Analysis in Metilene

Comparing Ventricular Methylomes to Non-Ventricular Tissue Methylomes

$ metilene_input.pl-in1 GSM1010978_Left_Ventricle.sorted, GSM1010988_Right_Ventricle.sorted, GSM983650_Left _Ventricle2.sorted-in2 GSM1010979_Thymus.sorted, GSM1010980_Ovary.sorted, GSM1010981_Adrenal_Gland.sorted, GSM1010983_Adipose_Tissue.sorted, GSM1010984_Gastric.sorted, GSM1010986_Psoas_Muscle.sorted, GSM1010987_Right_Atrium.sorted, GSM1010989_Sigmoid_Colon.sorted, GSM1112838_Brain_Hippocampus_Middle.sorted, GSM1127054_Breast_Myoepithelial_Cells.sorted, GSM916049_Adult_Liver.sorted, GSM916050_Brain_Hippocampus_Middle2.sorted, GSM983645_Sigmoid_Colon2.sorted, GSM983646_Small_Intestine.sorted, GSM983647_Lung.sorted, GSM983648_Aorta.sorted, GSM983649_Esophagus.sorted, GSM983651_Pancreas. sorted, GSM983652_Spleen.sorted-h1 Ventricles-h2 Tissues-out Metilene_Ventricles_Tissues.input & >Metilene_Ventricles_Tissues.input

General Script:

$ metilene_input.pl-in1<comma separated sorted bedgraph files of epigenomes from left and right ventricle tissues>-in2<comma separated sorted bedgraph files of epigenomes from non-ventricular tissues>-h1 Ventricles-h2 Tissues/Cells-out Metilene_Filename.input

Comparing Ventricular Methylomes to Hematopoietic Cell Methylomes

$ metilene_input.pl-in1 GSM1010978_Left_Ventricle.sorted, GSM1010988_Right_Ventricle.sorted, GSM983650_Left_Ventricle2.sorted-in2 EGAX00001086969_Neutrophil_venous.sorted, EGAX00001086971_Neutrophil_venous.sort ed, EGAX00001086972_Neutrophil_venous.sorted, EGAX00001097771_neutrophil_cord.sort ed, EGAX00001097776_Neutrophil_venous.sorted, EGAX00001128259_plasma_cell_bone.sorted, EGAX00001147725_macrophage_venous.sorted, EGAX00001195936_memory_B_venous.sorted, EGAX00001208464_erythroblast_cord.sorted, EGAX00001208466_CD4_alpha_beta_T_cell_venous.sorted, EGAX00001236255_inflammatory_macrophage_cord.sorted, EGAX00001236257_regulatory_T_cell_venous.sorted, EGAX00001236260_eosinophil_venous.sorted, GSM916052_CD34_Primary_Cells.sorted-h1 Ventricles-h2 Cells-out Metilene_Ventricles_Cells.input & >Metilene_Ventricles_Cells.input

General Script:

$ metilene_input.pl-in1<comma separated sorted bedgraph files of epigenomes from left and right ventricle tissues>-in2<comma separated sorted bedgraph files of epigenomes from hematopoietic cells>-h1 Ventricles-h2 Tissues/Cells-out Metilene_Filename.input

Filtering and Sorting DMRs by 10% to Obtain an Output File

Filtering and Sorting DMRs Between Ventricular and Non-Ventricular Tissues

$ metilene-M 25-m 4-d 0.1-t 4-f 1-a Ventricles-b Tissues-X 1-Y 1-v 0.7 Metilene_Ventricles_Tissues.input >Metilene_Ventricles_Tissues.output|sort-V-k1,1-k2,2n

Filtering and Sorting DMRs Between Ventricular Tissues and Hematopoietic Cells

$ metilene-M 25-m 4-d 0.1-t 4-f 1-a Ventricles-b Tissues-X 1-Y 1-v 0.7 Metilene_Ventricles_Cells.input >Metilene_Ventricles_Cells.output|sort-V-k1,1-k2,2n

Filtering the Output File to Obtain DMRs with a Methylation Difference of 50-80%

Filtering DMRs Between Ventricular and Non-Ventricular Tissues (Difference of 50%)

$ metilene_output.pl-q Metilene_Ventricles_Tissues.output-o Metilene_Ventricles_Tissues_Filtered-p 0.05-d 0.5-c 4-I 0-a Ventricles-b Tissues

Filtering DMRs Between Ventricular Tissues and Hematopoietic Cells (Difference of 80%)

$ metilene_output.pl-q Metilene_Ventricles_Cells.output-o Metilene_Ventricles_Cells_Filtered-p 0.05-d 0.8-c 4-I 0-a Ventricles-b Cells

Finding Common DMRs Between the Non-Ventricular Tissues and Hematopoietic Cells

$ bedtools intersect-a<Metilene_Ventricles_Tissues_Filtered.bedgraph>-b <Metilene_Ventricles_Cells_Filtered.bedgraph>

Primer Annealing Temperature Optimization

In order to determine the appropriate temperature that would allow for optimal primer annealing, a temperature gradient experiment was conducted. Each 200 μL PCR tube contained a total of 25 μL of reaction mixture. This mixture was comprised of 5 μL of 5× EpiMark® Hot Start Taq Reaction Buffer (New England BioLabs), 0.5 μl of 10 mM dNTP mix (Invitrogen), 0.5 μl of 10 μM forward primer, 0.5 μl of 10 μM reverse primer, 1.0 uL of 10 ng/μL bisulfite converted control human DNA (Qiagen), 0.125 μL of 5.00 U/mL EpiMark Hot Start Taq DNA Polymerase (New England BioLabs), and 17.38 μL of RNAse-free water to ensure the total volume was 25 uL. It should be noted that a master mix for each set of primers for a given DMR was created to reduce errors associated with pipetting volumes into each individual tube. Furthermore, the reagents were added in the order mentioned above. The tubes were then placed in the thermocycler and the PCR reaction was allowed to take place as follows: 3 minutes at 94° C., 45 seconds at 94° C., 45 seconds at one of eight temperatures between 55° C.-65° C., 1.5 minutes at 68° C., steps 2-4 were repeated 40×, 10 minutes at 68° C. followed by an infinite hold at 4° C. Products were visualized on a 3% agarose TAE gel using SYBR Green dye (Invitrogen) and a BioRad ChemiDoc Gel Imaging System.

PCR of Bisulfite-Converted DNA

Each 200 μL PCR tube contained 5 μL of 5× EpiMark Hot Start Taq Reaction Buffer (New England BioLabs), 0.5 μL of 10 mM dNTP mix (Invitrogen), 0.5 μL of 10 OA forward primer, 0.5 μL of 10 OA reverse primer, 1.0 μL of 10 ng/μL bisulfite-converted control human DNA (Qiagen), 0.125 μL of 5 U/mL EpiMark Hot Start Taq DNA Polymerase (New England BioLabs), and 17.38 μL of RNAse-free water for a final total volume of 25 μL. Tubes were then vortexed and centrifuged then placed in the thermocycler with the following program: 3 minutes at 94° C., 45 seconds at 94° C., 45 seconds at the determined Tm of the given primer, 1.5 minutes at 68° C., steps 2-4 were repeated 40×, 10 minutes at 68° C. followed by an infinite hold at 4° C.

[00164]Table 3. Publicly-available methylomes of both ventricular and non-ventricular
tissues and hematopoietic cells used for in silico DMR identification.

| | | |
|---|---|---|
| Tissue/cell type | Project | Accession number |
| Left ventricle | Roadmap | GSM1010978 |
| Left ventricle | Roadmap | GSM983650 |
| Right ventricle | Roadmap | GSM1010988 |
| Adipose tissue | Roadmap | GSM1010983 |
| Adrenal gland | Roadmap | GSM1010981 |
| Aorta | Roadmap | GSM983648 |
| Breast myoepithelial cells | Roadmap | GSM1127054 |
| Esophagus | Roadmap | GSM983649 |
| Gastric tissue | Roadmap | GSM1010984 |
| Hippocampus | Roadmap | GSM916050 |
| Hippocampus | Roadmap | GSM1112838 |
| Liver | Roadmap | GSM916049 |
| Lung | Roadmap | GSM983647 |
| Ovary | Roadmap | GSM1010980 |
| Pancreas | Roadmap | GSM983651 |
| Psoas muscle | Roadmap | GSM1010986 |
| Right atrium | Roadmap | GSM1010987 |
| Sigmoid colon | Roadmap | GSM983645 |
| Sigmoid colon | Roadmap | GSM1010989 |
| Small intestine | Roadmap | GSM983646 |
| Spleen | Roadmap | GSM983652 |
| Thymus | Roadmap | GSM1010979 |
| CD34+ cells | Roadmap | GSM916052 |
| Plasma cells (bone marrow) | Blueprint | EGAX00001128259 |
| Erythroblasts | Blueprint | EGAX00001208464 |
| Inflammatory macrophages | Blueprint | EGAX00001236255 |
| Neutrophils (cord blood) | Blueprint | EGAX00001097771 |
| Eosinophils | Blueprint | EGAX00001236260 |
| Macrophages (venous blood) | Blueprint | EGAX00001147725 |
| Memory B cells | Blueprint | EGAX00001195936 |
| Neutrophils (venous blood) | Blueprint | EGAX00001086969 |
| Neutrophils (venous blood) | Blueprint | EGAX00001086971 |
| Neutrophils (venous blood) | Blueprint | EGAX00001086972 |
| Neutrophils (venous blood) | Blueprint | EGAX00001097776 |
| Regulatory T cells (venous blood) | Blueprint | EGAX00001236257 |
| CD4+ cells (venous blood) | Blueprint | EGAX00001208466 |

| [00165] | Table 4. Putative aorta-specific DMRs identified including position, length in | | | | | |
|---|---|---|---|---|---|---|
| percents | irs, number of CpG sites within each DMR and mean methylation differences as a | | | | | |
| base pa | age compared to non-ventricle tissues | | | and hematopoietic cells. | | |
| | | DMR | | | Mean Methylation Difference (%) | |
| Chr | Start | End | Length | #CpGs | Non-aortic tissue | Hematopoietic |
| | Position | Position | (bp) | | | cells |
| 1* | 3,192,823 | 3,192,903 | 80 | 7 | -71.28 | -90.77 |
| 1 | 3,459,874 | 3,459,985 | 111 | 11 | -65.30 | -91.19 |
| 1 | 230,346,868 | 230,346,973 | 105 | 10 | -79.54 | -94.14 |
| 2 | 10,544,979 | 10,545,106 | 127 | 9 | -62.71 | -90.82 |
| 2* | 128,431,046 | 128,431,099 | 53 | 6 | -72.51 | -92.80 |
| 2 | 241,536,130 | 241,536,203 | 73 | 6 | -61.47 | -94.05 |
| 6 | 157,470,003 | 157,470,151 | 148 | 11 | -64.82 | -90.59 |
| 7 | 137.654.127 | 137.654.186 | 59 | 6 | -92.32 | -96.86 |
| 8 | 1,765,610 | 1,765,690 | 80 | 7 | 63.22 | 91.15 |
| 8* | 6,398,313 | 6,398,394 | 81 | 7 | -77.97 | -92.61 |
| 9 | 84,228,330 | 84,228,393 | 63 | 8 | -60.60 | -91.23 |

| 9* | 134,550,721 | 134,550,791 | 70 | 7 | -73.42 | -91.20 |
|---|---|---|---|---|---|---|
| 10 | 12,527,035 | 12,527,132 | 97 | 7 | -72.58 | -92.33 |
| 11** | 3,168,734 | 3,168,832 | 98 | 6 | -70.60 | -92.75 |
| 11 | 80,410,507 | 80,410,580 | 73 | 6 | -69.75 | -90.46 |
| 11 | 111,784,373 | 111,784,468 | 95 | 10 | -63.40 | -93.97 |
| 15* | 28,355,445 | 28,355,503 | 58 | 7 | -81.67 | -91.54 |
| 17 | 925,451 | 925,558 | 107 | 7 | -68.91 | -91.78 |
| 17 | 76,858,242 | 76,858,303 | 61 | 6 | -61.25 | -93.76 |
| 18** | 74,171,459 | 74,171,505 | 46 | 8 | -74.26 | -91.38 |
| 19* | 3,603,393 | 3,603,454 | 61 | 7 | -62.70 | -92.308 |
| 20** | 45,860,401 | 45,860,466 | 65 | 6 | 86.70 | -81.08 |
| 22** | 27,999,645 | 27,999,717 | 72 | 7 | -75.36 | -91.27 |
| 22 | 47,075,770 | 47,075,849 | 79 | 8 | -63.22 | -90.38 |
| [00166] | * These DMRs were selected for the first round of primer generation and | | | | | |
| testing. | | | | | | |

| | |
|---|---|
| [00167] | ** After primer generation and testing, these four DMRs were selected for |

complete validation.

| [00168]Table 5. Forward and reverse bisulfite PCR primer sequences designed using | | | | | | |
|---|---|---|---|---|---|---|
| MethPrimer for the DMRs and the associated melting temperatures. | | | | | | |
| Ch | Amplime | Forward primer | SE | Reverse primer | Tm | SE |
| r | r size | | Q | | (°C | Q |
| | (bp) | | ID | | ) | ID |
| | | | NO: | | | NO: |
| 11 | 127 | GGGTATTTAGTTATGA | 1 | CAAACCTATCTTTAATT | 55. | 2 |
| | | G-GGAATAATG | | T-CCACCC | 7 | |
| 18 | 120 | AGTTTAGGATTTGTGT | 3 | TAAAAAATATTACTATT | 55 | 4 |
| | | T-ATTTAGGA | | A-ACATCATAACAA | | |
| 20 | 139 | GGAGTAAAATGAATA | 5 | AAAAAACAAATACAAA | 58. | 6 |
| | | A-AATTTTTGTTGAGA | | A-AAACTACAAACC | 5 | |
| 22 | 122 | GTTGAGGAATTGGAGG | 7 | ACAAACTACTAAACAAA | 56. | 8 |
| | | -AAAATTAA | | -AAACACAA | 9 | |

| [00169] | Table 6. Correlation between aortic cfDNA levels for our candidate DMRs and | | | | | |
|---|---|---|---|---|---|---|
| elastin prope | rties within aortic regions | | of elevated wall shear stress. | | | |
| DMR | Elastin Area | | Elastin Fibre Thickness | | Inter-fibre Distance | |
| | R² | P | R² | P | R² | P |
| Chr 11 | 0.0006 | 0.95 | 0.18 | 0.17 | 0.28 | 0.064 |
| Chr 18 | 0.01 | 0.82 | 0.27 | 0.19 | 0.091 | 0.32 |
| Chr 20 | 0.21 | 0.37 | 0.23 | 0.34 | 0.017 | 0.7 |
| Chr 22 | 0025 | 0.66 | 0.19 | 0.15 | 0.26 | 0.074 |

| [00170] | Table 7. Correlation between aortic cfDNA levels for our candidate DMRs and | | | | | | | | | |
|---|---|---|---|---|---|---|---|---|---|---|
| protein le | vels within aortic | | ? regions of elevated wall shear stress. | | | | | | | |
| DMR | MMP-1 | | MMP-2 | | MMP-3 | | TGFp-1 | | TIMP-I | |
| | R² | P | R² | P | R² | P | R² | P | R² | P |
| 11 | 0.064 | 0.45 | 0.04 | 0.53 | 0.13 | 0.23 | 0.001 | 0.91 | 0.17 | 0.18 |
| 18 | 0.11 | 0.43 | 0.063 | 0.55 | 0.12 | 0.36 | 0.034 | 0.64 | 0.13 | 0.35 |
| 20 | 0.27 | 0.37 | 0.0022 | 0.92 | 0.14 | 0.41 | 0.15 | 0.39 | 0.0002 | 0.98 |
| 22 | 0.041 | 0.55 | 0.003 | 0.87 | 0.14 | 0.2 | 0.005 | 0.81 | 0.11 | 0.29 |

### REFERENCES

1. S. Verma, S. C. Siu, Aortic dilatation in patients with bicuspid aortic valve. N Engl J Med 370, 1920-1929 (2014).
2. A.-S. Sillesen et al., Prevalence of Bicuspid Aortic Valve and Associated Aortopathy in Newborns in Copenhagen, Denmark. JAMA 325, 561-567 (2021).
3. K. Huntington, A. G. Hunter, K. L. Chan, A prospective study to assess the frequency of familial clustering of congenital bicuspid aortic valve. J Am Coll Cardiol 30, 1809-1812 (1997).
4. M. Gago-Diaz et al., The genetic component of bicuspid aortic valve and aortic dilation. An exome-wide association study. J Mol Cell Cardiol 102, 3-9 (2017).
5. A. J. Barker et al., Bicuspid aortic valve is associated with altered wall shear stress in the ascending aorta. Circ Cardiovasc Imaging 5, 457-466 (2012).
6. A. J. Barker, M. Markl, P. W. M. Fedak, Assessing wall stresses in bicuspid aortic valve-associated aortopathy: Forecasting the perfect storm? J Thorac Cardiovasc Surg 156, 471-472 (2018).
7. P. W. Fedak et al., Clinical and pathophysiological implications of a bicuspid aortic valve. Circulation 106, 900-904 (2002).
8. P. W. Fedak et al., Vascular matrix remodeling in patients with bicuspid aortic valve malformations: implications for aortic dilatation. J Thorac Cardiovasc Surg 126, 797-806 (2003).
9. D. G. Guzzardi et al., Valve-Related Hemodynamics Mediate Human Bicuspid Aortopathy: Insights From Wall Shear Stress Mapping. J Am Coll Cardiol 66, 892-900 (2015).
10. M. A. Borger et al., The American Association for Thoracic Surgery consensus guidelines on bicuspid aortic valve-related aortopathy: Full online-only version. J Thorac Cardiovasc Surg 156, e41-e74 (2018).
11. R. Erbel et al., 2014 ESC Guidelines on the diagnosis and treatment of aortic diseases: Document covering acute and chronic aortic diseases of the thoracic and abdominal aorta of the adult. The Task Force for the Diagnosis and Treatment of Aortic Diseases of the European Society of Cardiology (ESC). Eur Heart J 35, 2873-2926 (2014).
12. J. H. Wasfy, K. Armstrong, C. E. Milford, T. M. Sundt, Bicuspid aortic disease and decision making under uncertainty—The limitations of clinical guidelines. Int J Cardiol 181, 169-171 (2015).
13. A. Della Corte et al., Risk Stratification in Bicuspid Aortic Valve Aortopathy: Emerging Evidence and Future Perspectives. Curr Probl Cardiol, (2019).
14. A. Della Corte et al., Surgical treatment of bicuspid aortic valve disease: knowledge gaps and research perspectives. J Thorac Cardiovasc Surg 147, 1749-1757, 1757 e1741 (2014).
15. A. K. Maredia, S. C. Greenway, S. Verma, P. W. M. Fedak, Bicuspid aortic valve-associated aortopathy: update on biomarkers. Curr Opin Cardiol, (2017).
16. Y. M. Lo et al., Presence of fetal DNA in maternal plasma and serum. Lancet 350, 485-487 (1997).
17. Y. M. Lo et al., Presence of donor-specific DNA in plasma of kidney and liver-transplant recipients. Lancet 351, 1329-1330 (1998).
18. S. Grabuschnig et al., Putative Origins of Cell-Free DNA in Humans: A Review of Active and Passive Nucleic Acid Release Mechanisms. Int J Mol Sci 21, (2020).
19. V. Gedvilaite, D. Schveigert, S. Cicenas, Cell-free DNA in non-small cell lung cancer. Acta Med Litu 24, 138-144 (2017).
20. R. Lehmann-Werman et al., Identification of tissue-specific cell death using methylation patterns of circulating DNA. Proc Natl Acad Sci USA 113, E1826-1834 (2016).
21. S. K. Pattar, S. C. Greenway, Circulating nucleic acids as biomarkers for allograft injury after solid organ transplantation: current state-of-the-art. Transplant Research and Risk Management Volume 11, 17-27 (2019).
22. D. Prasher, S. C. Greenway, R. B. Singh, The impact of epigenetics on cardiovascular disease. Biochem Cell Biol 98, 12-22 (2020).
23. G. Egger, G. Liang, A. Aparicio, P. A. Jones, Epigenetics in human disease and prospects for epigenetic therapy. Nature 429, 457-463 (2004).
24. H. I. Michelena et al., Bicuspid aortic valve aortopathy in adults: Incidence, etiology, and clinical significance. Int J Cardiol 201, 400-407 (2015).
25. Y. Nakamura et al., The analysis of ascending aortic dilatation in patients with a bicuspid aortic valve using the ratio of the diameters of the ascending and descending aorta. J Cardiothorac Surg 9, 108 (2014).
26. L. Adamo, A. C. Braverman, Surgical threshold for bicuspid aortic valve aneurysm: a case for individual decision-making. Heart 101, 1361-1367 (2015).
27. M. Nataatmadja et al., Abnormal extracellular matrix protein transport associated with increased apoptosis of vascular smooth muscle cells in marfan syndrome and bicuspid aortic valve thoracic aortic aneurysm. Circulation 108 Suppl 1,11329-334 (2003).
28. J. Liu, J. A. Shar, P. Sucosky, Wall Shear Stress Directional Abnormalities in BAV Aortas: Toward a New Hemodynamic Predictor of Aortopathy? Front Physiol 9, 993 (2018).
29. S. A. Mohamed et al., Inhibition of caspase-3 differentially affects vascular smooth muscle cell apoptosis in the concave versus convex aortic sites in ascending aneurysms with a bicuspid aortic valve. Ann Anat 192, 145-150 (2010).
30. J. Schindelin et al., Fiji: an open-source platform for biological-image analysis. Nature Methods 9, 676-682 (2012).
31. A. Kundaje et al., Integrative analysis of 111 reference human epigenomes. Nature 518, 317-330 (2015).
32. H. G. Stunnenberg, C. International Human Epigenome, M. Hirst, The International Human Epigenome Consortium: A Blueprint for Scientific Collaboration and Discovery. Cell 167, 1145-1149 (2016).
33. F. Juhling et al., metilene: fast and sensitive calling of differentially methylated regions from bisulfite sequencing data. Genome Res 26, 256-262 (2016).
34. L. C. Li, R. Dahiya, MethPrimer: designing primers for methylation PCRs. Bioinformatics 18, 1427-1431 (2002).
35. S. Andrews, in http://www.bioinformatics.babraham.ac.uk/projects/fastqc/. (2018).
36. H. H. Sievers, C. Schmidtke, A classification system for the bicuspid aortic valve from 304 surgical specimens. J Thorac Cardiovasc Surg 133, 1226-1233 (2007).

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A method of determining the risk of a human subject with congenital BAV having BAV-associated aortopathy or developing BAV-associated aortopathy, comprising:
obtaining a cell free DNA (cfDNA) sample from the subject,
measuring the level of methylation of a methylation marker that is associated with BAV-associated aortopathy in the cfDNA sample,
optionally comparing the level of methylation of the methylation maker in the sample of the subject with a comparator control,
wherein disproportionate hypomethylation of the methylation marker in the cfDNA sample would suggest that the subject is at risk of developing BAV-associated aortopathy.

2. The method of claim 1, wherein the methylation marker comprises a differentially methylated region (DMR) on Chr 11.

3. The method of claim 2, wherein the DMR on Chr 11 comprises position Chr 11:3,168,734-3,168,832.

4. The method of any one of claims 1, wherein the step of measuring the cfDNA for the presence of one or more methylation markers comprises sequencing the cfDNA.

5. The method of claim 4, wherein sequencing comprises bisulfite sequencing.

6. The method of claim 5, wherein bisulfite sequencing is carried out using a forward primer (GGGTATTTAGTTATGAGGGAATAATG; SEQ ID NO:1) and a reverse primer (CAAACCTATCTTTAATTTCCACCC; SEQ ID NO:2).

7. The method of claim 5, wherein the sequencing comprises droplet digital PCR assay.

8. A kit for determining the risk of a human subject with congenital BAV having BAV-associated aortopathy or developing BAV-associated aortopathy, comprising:
a forward primer (GGGTATTTAGTTATGAG-GGAATAATG; SEQ ID NO: 1) and a reverse primer (CAAACCTATCTTTAATTTCCACCC; SEQ ID NO:2), optionally a container, and optionally instructions for the use thereof.


SEQUENCE LISTING (8 sequences)

SEQ ID NO: 1 - DNA, 26 bp, synthetic construct
  source 1..26: mol_type = other DNA
gggtatttagttatgagggaataatg

SEQ ID NO: 2 - DNA, 24 bp, synthetic construct
  source 1..24: mol_type = other DNA
caaacctatctttaatttccaccc

SEQ ID NO: 3 - DNA, 25 bp, synthetic construct
  source 1..25: mol_type = other DNA
agtttaggatttgtgttatttagga

SEQ ID NO: 4 - DNA, 30 bp, synthetic construct
  source 1..30: mol_type = other DNA
taaaaaatattactattaacatcataacaa

SEQ ID NO: 5 - DNA, 30 bp, synthetic construct
  source 1..30: mol_type = other DNA
ggagtaaaatgaataaaatttttgttgaga

SEQ ID NO: 6 - DNA, 29 bp, synthetic construct
  source 1..29: mol_type = other DNA
aaaaaacaaatacaaaaaaactacaaacc

SEQ ID NO: 7 - DNA, 24 bp, synthetic construct
  source 1..24: mol_type = other DNA
gttgaggaattggaggaaaattaa

SEQ ID NO: 8 - DNA, 25 bp, synthetic construct
  source 1..25: mol_type = other DNA
acaaactactaaacaaaaaacacaa